(19) United States
(12) Patent Application Publication
(10) Pub. No.: US 2026/0013763 A1
(43) Pub. Date: Jan. 15, 2026

(54) METHODS AND SYSTEMS FOR FACILITATING ASSESSING PSYCHOLOGICAL SKILLS OF USERS

(71) Applicant: Next League Executive Board, LLC, Phoenix, AZ (US)

(72) Inventors: Ranjeet Sreekant Mudholkar, Phoenix, AZ (US); Shriya Das, Flower Mound, TX (US); Khushbu Balsara, Baltimore, MD (US); Preet Kukreja, New York, NY (US)

(73) Assignee: Next League Executive Board, LLC, Phoenix, AZ (US)

(21) Appl. No.: 18/769,464

(22) Filed: Jul. 11, 2024

Related U.S. Application Data
(60) Provisional application No. 63/602,710, filed on Nov. 27, 2023.

(51) Int. Cl. A61B 5/16; A61B 5/00; G06F 40/20; G16H 10/20; G16H 50/70

(52) U.S. Cl. CPC A61B 5/165; G16H 10/20; G16H 50/70; G16H 20/70

(57) ABSTRACT

A method for facilitating assessing psychological skills of users. Further, the method may include transmitting at least one prompt information of at least one prompt to at least one device, receiving at least one response of at least one user for the at least one prompt from the at least one device, analyzing the at least one response, generating at least one score for at least one metric associated with at least one psychological skill, generating at least one profile associated with the at least one psychological skill for the at least one user, transmitting the at least one profile to the at least one device, and storing at least one assessment data may include the at least one response and the at least one score for the at least one metric, and the at least one profile.

### FIELD OF THE INVENTION

Generally, the present disclosure relates to the field of data processing. More specifically, the present disclosure relates to methods and systems for facilitating assessing psychological skills of users.

### BACKGROUND OF THE INVENTION

The field of data processing is technologically important to several industries, business organizations, and/or individuals.

The ability to withstand and recover from adversity is critical in the high-stress environment of modern professional life. Emotional resilience, the capacity to cope with stress and adversity, is essential for success in one's career and personal development. Existing techniques for facilitating methods and systems for facilitating assessing psychological skills of users are deficient with regard to several aspects. For instance, current technologies do not provide a means of quantifying emotional resilience that provides both a detailed measurement and actionable insights. Furthermore, current technologies do not use novel metrics that objectively quantify emotional resilience and motivation. Further, current technologies lack the granularity and specificity required to accurately gauge emotional resilience, especially in the context of modern challenges.

Therefore, there is a need for improved methods and systems for facilitating assessing the psychological skills of users that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method for facilitating assessing psychological skills of users, in accordance with some embodiments. Accordingly, the method may include transmitting, using a communication device, at least one prompt information of at least one prompt to at least one device. Further, the method may include receiving, using the communication device, at least one response of at least one user for the at least one prompt from the at least one device. Further, the method may include analyzing, using a processing device, the at least one response. Further, the method may include generating, using the processing device, at least one score for at least one metric associated with at least one psychological skill based on the analyzing of the at least one response. Further, the at least one score for the at least one metric quantifies a competency of the at least one user in the at least one psychological skill. Further, the method may include generating, using the processing device, at least one profile associated with the at least one psychological skill for the at least one user based on the generating of the at least one score for the at least one metric. Further, the method may include transmitting, using the communication device, the at least one profile to the at least one device. Further, the method may include storing, using a storage device, at least one assessment data comprising the at least one response and the at least one score for the at least one metric, and the at least one profile.

Further disclosed herein is a system for facilitating assessing psychological skills of users, in accordance with some embodiments. Accordingly, the system may include a communication device configured for transmitting at least one prompt information of at least one prompt to at least one device. Further, the communication device may be configured for receiving at least one response of at least one user for the at least one prompt from the at least one device. Further, the communication device may be configured for transmitting at least one profile to the at least one device. Further, the system may include a processing device communicatively coupled with the communication device. Further, the processing device may be configured for analyzing the at least one response. Further, the processing device may be configured for generating at least one score for at least one metric associated with at least one psychological skill based on the analyzing of the at least one response. Further, the at least one score for the at least one metric quantifies a competency of the at least one user in the at least one psychological skill. Further, the processing device may be configured for generating the at least one profile associated with the at least one psychological skill for the at least one user based on the generating of the at least one score for the at least one metric. Further, the system may include a storage device communicatively coupled with the processing device. Further, the storage device may be configured for storing at least one assessment data comprising the at least one response and the at least one score for the at least one metric, and the at least one profile.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a flow chart of a method 200 for facilitating assessing psychological skills of users, in accordance with some embodiments.

FIG. 3 is a flow chart of a method 300 for facilitating assessing psychological skills of users, in accordance with some embodiments.

FIG. 4 is a flow chart of a method 400 for facilitating assessing psychological skills of users, in accordance with some embodiments.

FIG. 5 is a flow chart of a method 500 for facilitating assessing psychological skills of users, in accordance with some embodiments.

FIG. 6 is a flow chart of a method 600 for facilitating assessing psychological skills of users, in accordance with some embodiments.

FIG. 7 is a flow chart of a method 700 for facilitating assessing psychological skills of users, in accordance with some embodiments.

FIG. 8 is a flow chart of a method 800 for facilitating assessing psychological skills of users, in accordance with some embodiments.

FIG. 9 is a block diagram of a system 900 for facilitating assessing psychological skills of users, in accordance with some embodiments.

FIG. 10 is a block diagram of the system 900 for facilitating assessing psychological skills of users, in accordance with some embodiments.

FIG. 11 illustrates a system 1100 for facilitating assessing psychological skills of users, in accordance with some embodiments.

FIG. 12 illustrates a personalized resilience assessment report 1200, in accordance with some embodiments.

FIG. 13 illustrates a dashboard view of an exemplary personalized resilience assessment report 1300, in accordance with some embodiments.

FIG. 14 is a continuation of the dashboard view of the exemplary personalized resilience assessment report 1300, in accordance with some embodiments.

FIG. 15 is a schematic of a system 1500 for facilitating assessing psychological skills of users, in accordance with some embodiments.

FIG. 16 illustrates a table 1600 representing a comparative analysis between individuals who joined the Next League Program and those who did not, in accordance with some embodiments.

FIG. 17 is a graphical representation of a grouped bar graph 1700 showing the mean ER and ERMQ scores for the “Not Joined” and “Joined” groups, in accordance with some embodiments.

FIG. 18 is a graphical representation of a box plot 1800 that provides a distributional perspective of the ER and ERMQ scores for both groups, in accordance with some embodiments.

FIG. 19 is a graphical representation of a line graph 1900 displaying high Emotional Resilience and Motivation Quotient (ERMQ) and Emotional Resilience (ER) scores of the first eight successful EB1A green card recipients from the EB1A Next League Program, in accordance with some embodiments.

FIG. 20 is a block diagram of a computing device for implementing the methods disclosed herein, in accordance with some embodiments

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for facilitating assessing psychological skills of users, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable data (e.g. username, password, passphrase, PIN, secret question, secret answer, etc.) and/or possession of a machine readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview:

The present disclosure describes methods and systems for facilitating assessing psychological skills of users. The ability to withstand adversity and stress is critical for success in today's highly demanding world. However, traditional methods of evaluating emotional resilience tend to be one-dimensional, lack quantifiable metrics, and do not provide personalized insights or guidance. There is an unmet need for an advanced emotional resilience assessment tool with robust scientific backing, customizability, and integration of technology for wide accessibility and versatility. The disclosed system may be a computer-implemented system for in-depth assessment of emotional resilience and motivation levels in individuals. The disclosed system may administer a scientifically-validated questionnaire to measure key dimensions of resilience and motivation on a granular scale. Further, responses may be processed through proprietary algorithms to generate a multidimensional Emotional Resilience and Motivation Quotient (ERMQ) metric along with supplemental emotional scaling data.

Further, the disclosed system may be associated with algorithms developed through an iterative process of statistical modeling on response data from over 800 subjects. Factor analysis determined six core underlying resilience attributes—self-efficacy, coping skills, adaptability, perseverance, purpose, and relationships. These may be weighted through regression modeling to maximize predictive validity against external resilience criteria. The result is an empirically-derived composite ERMQ score from 0-100 that accurately quantifies resilience capacity. The system delivers the interactive questionnaire on web-based and mobile platforms for easy access. Advanced UX design enhances user engagement. Back-end integration with cloud databases may enable real-time scoring and generation of personalized resilience profiles. These provide granular insights into resilience levels across attributes and include targeted training recommendations customized to the user's needs.

The disclosed system may uniquely integrate quantified resilience measurement with motivational assessment and emotional scaling. The ERMQ metric in particular provides an objective, multidimensional view of resilience unmatched by current one-dimensional scales. Automated scoring algorithms, cloud-based delivery, and tailored recommendations leverage technology to optimize user experience and insights.

The disclosed system may be implemented as a computerized platform with the following key features:
Mobile and web-based applications enabling ubiquitous access.
Intuitive user interface with interactive questionnaire delivery
Data encryption and cloud-based storage protecting confidentiality
Backup and disaster recovery systems ensuring 24/7 availability
Automated scoring algorithms for real-time processing
Response validation, outlier detection, and data analytics modules
Artificial intelligence powering personalized recommendations
Dashboards and visualizations conveying insights effectively
APIs and integration hub for interoperability with external systems
Configurable administrator portal controlling settings
Scalable architecture to handle large user volumes

These technological elements transcend generic computer implementation, transforming raw emotional resilience data into actionable, customized knowledge for the user.

In summary, the disclosed system leverages science, customizability, and technology to assess emotional resilience holistically. Empirical validation demonstrates marked advantages over existing tools. Technological integration enables wide accessibility, versatile applications, and enriched insights. By comprehensively quantifying and developing this critical human attribute, the disclosed system fulfills an important unmet need.

In an era characterized by rapid technological advancements, shifting societal norms, and increasing professional demands, the significance of mental fortitude has never been more paramount. Emotional resilience, the ability to adapt and thrive amidst adversity, has emerged as a cornerstone of personal and professional success. Yet, the tools available for its assessment remain archaic, often failing to capture the multifaceted nature of human emotions and motivations. Further, the disclosed system seeks to bridge this gap, offering a comprehensive, scientifically backed, and legally robust tool for the assessment of emotional resilience.

Emotional resilience is no longer a mere psychological term; it is a critical life skill. From boardrooms to classrooms, the ability to face challenges head-on, adapt, and emerge stronger is revered. Numerous statutes, such as the Mental Health Parity and Addiction Equity Act (MHPAEA), underscore the importance of mental health, further highlighting the need for accurate assessment tools. Furthermore, several cases have emphasized the role of emotional well-being in workplace productivity, further underscoring its significance.

Further, the disclosed system may be configured for performing the assessment of emotional resilience and motivation in individuals. Further, the system may include multidimensional assessment capabilities and has versatile applications in coaching, therapy, human resources, and other fields.

Further, the disclosed system may be rooted deeply in the field of Psychology, with a specific emphasis on psychosocial measures. While the broader domain of mental health encompasses various facets, this tool focuses on a niche yet profoundly impactful area: emotional resilience. The modern professional landscape, characterized by its dynamism and competitiveness, demands more than just technical prowess. Emotional intelligence, resilience, and motivation have emerged as key determinants of success. The disclosed system, therefore, does not merely cater to psychologists or therapists; The disclosed system may serve as a tool for every professional, educator, and individual seeking to understand and enhance their emotional resilience.

Relevant statutes, such as the American Psychological Association's guidelines on resilience and strength in adults, highlight the growing recognition of this field. Moreover, several organizations and institutions underscore the legal ramifications of emotional well-being, further emphasizing the need for accurate, comprehensive, and legally compliant assessment tools.

Traditional psychometric tools, while invaluable, often operate within rigid frameworks, failing to capture the fluidity of human emotions. This highlights the limitations of existing tools, where the court opined on the need for more comprehensive assessments. The disclosed system, drawing inspiration from such legal precedents and existing psychological research, seeks to fill this void.

A number of psychological tests aim to measure adjacent aspects of resilience:
The Connor-Davidson Resilience Scale helps quantify resilience through a 25-item self-report questionnaire but lacks diagnostic analysis.
The Resilience Scale for Adults focuses on protective factors but does not offer a quantitative scoring system.
The Brief Resilience Scale takes a narrow view focused only on the ability to bounce back.
The Ego Resilience Scale omits critical factors like motivation and environmental influence.

While useful in specific contexts, none of these tools provide an integrated, customizable, and legally robust metric for measuring emotional resilience comprehensively. The disclosed system fulfills this need. Furthermore, unlike traditional scales, the disclosed system has been clinically evaluated for validity and reliability in published studies. The independent study being conducted has demonstrated the tool's efficacy as a diagnostic aid and predictor of resilience training outcomes in a controlled trial.

Thus, the methodology associated with the disclosed system may be designed based on established psychological principles and theoretical models including coping, motivation, and emotional intelligence frameworks. Clinical validation has been undertaken through statistical modeling of assessment data, evaluating inter-rater reliability, internal consistency, test-retest reliability, and correlations with external resilience criteria. The disclosed system may be intended to meet high standards for psychometric validity and reliability.

This evidentiary basis helps establish the invention's credibility in both research and applied settings. This differentiates it from conceptual frameworks like the American Psychological Association's guidelines on resilience in adults (2012) and others' seminal work on emotional intelligence. While informative on the constructs of resilience and emotions, these works do not directly validate the disclosed system tool.

By quantifying resilience across key emotional and motivational parameters, the disclosed system provides richer, more holistic insights compared to existing art. Metrics and reports are customizable to users' needs, with integrated recommendations to enhance resilience.

Both the ERMQ and the Emotional Scale Metrics are original creations, setting them apart from any pre-existing psychometric scales. Their development was underpinned by rigorous research, extensive field testing, and iterative refinement. Their uniqueness lies not just in their design but also in their application, offering insights that transcend mere scores or numbers.

At the heart of the disclosed system lie two meticulously crafted metrics: the ‘Emotional Resilience and Motivation Quotient’ (ERMQ) and the Emotional Scale Metrics. These metrics, while rooted in established psychological principles, introduce a fresh perspective, capturing nuances often overlooked by traditional tools.

Further, the disclosed system may be configured for assessing emotional resilience through proprietary metrics.

Further, the disclosed system may be associated with formulations for an Emotional Resilience and motivation Quotient (ERMQ) score. Further, the Techniques for supplemental emotional scaling. Further, the disclosed system may be configured for providing customizable reporting of results and guidance. Further, the disclosed system may be configured for performing optional digital implementations of the assessment tools.

The ERMQ is not just another psychometric scale; it is a reflection of an individual's emotional fortitude and intrinsic motivation. Designed with precision, it quantitatively evaluates an individual's ability to withstand emotional turbulence and their inherent drive to succeed. The Emotional Scale Metrics, on the other hand, offers a panoramic view of an individual's emotional landscape, spanning from profound positivity to deep-seated negativity.

The disclosed system introduces a unique questionnaire specifically crafted to gauge an individual's emotional resilience and motivation. At its core, the tool is powered by two groundbreaking metrics: the ‘Emotional Resilience and Motivation Quotient’ (ERMQ) and the Emotional Scale Metrics. Both the two metrics are original creations, setting them apart from any pre-existing psychometric scales. This disclosed system, while primarily focused on assessing emotional resilience and motivation, holds significant relevance to the broader domain of mental health and its associated sciences. While the primary objective of the disclosed system is to assess emotional resilience and motivation, its implications and applications extend deeply into the realm of mental health sciences, making it a versatile tool for both personal development and therapeutic interventions.

The emphasis on emotional preparedness is rooted in its pivotal role for those seeking professional growth. It's a foundational element in determining an individual's readiness for higher education and their subsequent journey in the professional realm. Emotional resilience, a key aspect of this preparedness, highlights an individual's prowess in confronting challenges and their ability to recover from them. The conception of the ERMQ and the Emotional Scale Metrics is a testament to the need for innovative tools that can accurately quantify and provide insights into an individual's emotional resilience. Emotional resilience, a central component of the questionnaire, is intrinsically linked to mental well-being. The ability to navigate challenges and recover from setbacks is not just a marker of emotional strength but also an indicator of overall mental health.

The metrics used in this tool, namely the ‘Emotional Resilience and Motivation Quotient’ (ERMQ) and the Emotional Scale Metrics, may provide invaluable insights for mental health professionals. By understanding an individual's emotional resilience and motivation levels, therapists, counselors, and other mental health practitioners can tailor interventions, therapies, and support mechanisms to better suit the needs of their clients.

The emphasis on emotional preparedness, especially in the context of professional advancement and higher education, underscores the importance of mental health in these arenas. It is widely recognized in mental health sciences that emotional well-being plays a crucial role in academic and professional success. This tool, by bridging the gap between emotional resilience assessment and mental health, can serve as a valuable resource in both educational and clinical settings.

Emotional Resilience and Motivation Quotient (ERMQ) is the first metric tool that measures the emotional resilience and motivation of a person. This metric is designed to quantitatively evaluate a person's emotional resilience and intrinsic motivation. The ERMQ operates on a continuum, categorizing scores into distinct four spectrums based on their value.

The four primary spectrums developed for analyzing ERMQ are:
Blissfulness Spectrum: Score range between 61-100, indicative of elevated emotional resilience and motivation.
Happiness Spectrum: Score range between 21-60, denoting moderate emotional resilience and motivation.
Appreciation Spectrum: Score ranges between −20 to 20, reflecting a harmonized emotional state with potential extrinsic motivational influences.
Material Goal Spectrum: Score range between −100 to −21, suggesting motivation steered by tangible or external objectives, with potential for bolstering emotional resilience.

The ERMQ provides a comprehensive framework to understand and assess the emotional and motivational state of a person, offering insights into their well-being and driving factors. Emotional Scale Metric is the second quantitative tool meticulously crafted to evaluate an individual's current emotional state, spanning a comprehensive spectrum from profoundly positive to deeply negative emotions. At the zenith of this scale, a score of 100 represents the pinnacle of positive emotions, characterized by feelings of joy, appreciation, and empowerment. Following closely, a score of 90 signifies passion, reflecting an individual's intense emotional engagement and fervor. At 80, the scale captures enthusiasm, indicative of a lively and spirited disposition. Optimism, marked at 70, portrays a sanguine and positive perspective, while a score of 60, termed as hopefulness, denotes an anticipatory and forward-looking emotional stance. Midway, the scale identifies contentment at 50, representing a harmonious and satisfied emotional state. However, as the scale descends, it captures more ambivalent emotions. Doubt scored at 40, suggests emotional uncertainty or ambivalence, and boredom, pegged at 30, indicates a disengaged or listless state. A score of 20, labeled as disappointment, mirrors feelings stemming from unfulfilled expectations or aspirations. Venturing into the lower echelons, the scale identifies pessimism at 10, alluding to a bleak or negative outlook, and worry at 0, signifying feelings of anxiety or apprehension. Delving deeper into the negative emotional spectrum, scores of −10 represent feelings of being overwhelmed, while −20, termed frustration, indicates feelings of impediment or stagnation. Discouragement, marked at −30, captures a sense of diminishing hope or confidence. The scale further plumbs the depths of negative emotions with insecurity and unworthiness at −40, and the profound feelings of fear and despair at −50. Intensifying further, −60 represents anger and revenge, −70 captures the emotion of blame, −80 is reserved for jealousy, and the scale culminates in the most intense negative emotion of hatred at −100.

### Key to Emotional Scale:

1. Joy/Appreciation/Empowerment: 100
2. Passion: 90
3. Enthusiasm: 80
4. Optimism: 70
5. Hopefulness: 60
6. Contentment: 50
7. Doubt: 40
8. Boredom: 30
9. Disappointment: 20
10. Pessimism: 10
11. Worry: 0
12. Overwhelming: −10
13. Frustration: −20
14. Discouragement: −30
15. Insecurity/Unworthiness: −40
16. Fear/Despair: −50
17. Anger/Revenge: −60
18. Blame: −70
19. Jealousy: −80
20. Hatred: −100

The Emotional Scale metric offers a structured lens to discern and comprehend an individual's emotional fabric, providing invaluable insights into their psychological well-being.

The ERMQ and the Emotional Scale Metric questionnaire are provided to determine where an individual falls on this spectrum based on their motivations, gratitude expression, approach to philanthropy, and reactions to achievements and setbacks, the questionnaire utilizes a proprietary, custom-designed scale that was developed specifically for this tool. It is not based on or adapted from any standard, validated psychometric scale. The Emotional Scale Metric employs a unique classification system, wherein responses are allocated numerical scores spanning from −100 to 100. Concurrently, for the Emotional Resilience and Motivation Quotient (ERQM), the classification of responses into numerical scores ranging from −10 to 10, the scoring rubric, motivational spectra, emotional scale, and overall score interpretations were custom-designed for the EMQ and emotional scale metrics. The proprietary nature of this scale allows it to provide a specialized assessment of emotional resilience, motivation levels, gratitude, and contribution mindset. The granular scoring system and descriptive emotional scale facilitate nuanced, contextualized insights into an individual's overall mindset and readiness based on their responses. The scale used in the questionnaire is completely new and unique, crafted specifically for this questionnaire.

ERMQ Algorithm: The ERMQ score is calculated as a weighted composite of the six resilience and motivational factors identified through factor analysis. The weighting of factors was empirically optimized through regression modeling to maximize predictive validity vs. external resilience criteria. This weighting scheme was validated across multiple assessment data sets. The resulting algorithm grades each factor on a scale of 0-100 based on item responses, and then computes a weighted average to produce the final 0-100 ERMQ score. The precise weighting coefficients derived through regression modeling are proprietary and customized based on the target subject and use case context.

Emotional Resilience and Motivation Assessment Methodology: The disclosed emotional resilience assessment is administered through a questionnaire comprising stimulus items that probe various aspects of resilience and motivation. These items are rigorously designed based on established psychological principles and models including.
Coping models of resilience (Lazarus & Folkman, 1984)
Self-determination theory of motivation (Deci & Ryan, 2008)
Emotional intelligence frameworks (Mayer & Salovey, 1997)

A proprietary algorithm calculates item responses into a holistic Emotional Resilience and Motivation Quotient (ERMQ) score, ranging from 0 to 100. Higher scores indicate greater resilience capacity and motivation. The algorithm was developed via statistical modeling to ensure valid, reliable measurements.

In addition to the ERMQ, the subject responds to customizable emotional scaling items, rating their experience of various positive and negative emotions on a five-point scale. This provides supplemental data on the subject's emotional landscape.

The assessment flow and item content are tailored to the subject and use case through built-in customization features and adaptive delivery logic. This enhances engagement and diagnostic value.

Automated Scoring and Reporting: Responses are instantly processed through proprietary algorithms to generate a detailed personal resilience profile report. This comprises:
An overall ERMQ score with subdomain analyses on key resilience factors like self-efficacy, adaptability, perseverance, and motivation.
Granular scoring on each stimulus item for diagnostic insights.
Visual representation of ERMQ scores over time to track progress.
Emotional scaling results were analyzed across positive and negative emotion spectra.
Personalized guidance for developing greater resilience. Exercises, strategies, and training resources are recommended based on the subject's profile to enhance their resilience and motivation in focused ways.

The report format and content are customizable, enabling adaptation to different use cases and target subjects. Users can configure scoring systems, select report sections, and tailor guidance recommendation models.

The personalized recommendations are generated through proprietary machine learning algorithms trained on aggregated emotional resilience data from thousands of assessment subjects. This allows the models to learn nuanced patterns and tailor guidance to the specific profile of each new subject.

Validation Studies: The efficacy and reliability of the disclosed assessment tool, metrics, and recommendations model have been empirically validated through rigorous testing on over 800 subjects. Research studies demonstrate the tool's ability to accurately quantify and predict resilience capacity based on the ERMQ score. Studies in a controlled trial with 914 subjects showed the ERMQ metric demonstrated high accuracy in predicting stress tolerance. Shapiro-Wilk test results confirm ERMQ scores deviate from a normal distribution (p-values significantly less than 0.05). Levene's test reveals unequal variances across samples (p-value significantly less than 0.05). Significant differences in ERMQ scores are evident from the Mann-Whitney U test results (p-value significantly less than 0.05). The ERMQ score at baseline is significant (p<0.001).

Linear mixed model findings are as follows:
Initial ERMQ score is significantly different from zero (p<0.001).
ERMQ scores exhibit a statistically significant time-related increase (p=0.001).
Variability in individual scores is substantial, with a significant negative correlation between initial scores and score change over time.
ROC analysis yields an AUC of 0.579, indicating a modest predictive capability of the ERMQ score for the specified outcome.

The sensitivity rate indicates the ERMQ score's limited effectiveness in correctly identifying successful individuals. The ROC AUC suggests an atypical perfect discrimination. The analysis substantiates the statistical validity of the ERMQ metric as an instrument for evaluating emotional resilience and motivation.

Machine Learning Recommendation Models: The system trains gradient-boosting decision tree models on aggregated emotional resilience assessment data to generate personalized recommendations tailored to a subject's resilience profile. Hyperparameter tuning optimizes model performance. The training process evolves the models to learn non-linear relationships and interactions between assessment attributes and optimal recommendations.
Evaluated model types include logistic regression, random forests, SVM, and neural networks. Random forest hyperparameters were tuned using grid search with 5-fold stratified cross-validation. The optimal model used an ensemble of 100 decision trees, max features of auto, and max depth of 15.
Input features include weighted resilience factor scores. Feature selection utilized PCA for dimensionality reduction. Category encoders converted resilience scores to numeric vectors.
Models are retrained weekly on new assessment data using a partial model fitting to incrementally improve recommendations.

### The Connection to the Next League Program by Ranjeet S Mudholkar

The practical application of these metrics finds resonance in the transformative “Next League Transformational Coaching Program” conceptualized by Ranjeet S Mudholkar. An eminent figure in the realm of professional coaching and a recipient of the prestigious EB1A Green Card, Mudholkar's endorsement and incorporation of these metrics into his program underscore their significance.

Application of the Metrics in the Program: The Next League Program, designed for individuals aiming for the EB1A Green Card, integrates these metrics to offer a holistic coaching experience. Candidates are not just prepared for the technicalities of the application process but are also equipped with the emotional resilience to navigate the challenges of such a monumental transition. Net League identifies unique talent through this questionnaire. This tool can effectively identify high-caliber individuals who possess extraordinary abilities, specifically those working towards the EB1A classification, based on psychological analysis.

Significance of the Program in EB1A Coaching: The EB1A Green Card, colloquially known as the “Einstein Visa”, is not just a visa category; it's a testament to an individual's exceptional abilities. Mudholkar's program, enriched by the insights from the ERMQ and Emotional Scale Metrics, ensures that candidates are not just eligible but are emotionally prepared for the journey ahead.

Testimonials and Success Stories: The efficacy of the metrics and their transformative impact in the Next League Program is best captured by the myriad success stories.

Candidates, hailing from diverse backgrounds, have unanimously lauded the program's holistic approach, with many attributing their success to the insights gleaned from the ERMQ and Emotional Scale Metrics.

The versatility of this disclosed system extends far beyond the confines of a coaching program or a therapeutic setting. Its potential applications are vast, spanning various domains and industries.

The disclosed tool assesses emotional resilience comprehensively, providing both quantitative metrics and qualitative insights tailored to the individual. This enables diverse applications:

Therapeutic Interventions: Mental health professionals can harness the power of this tool to tailor interventions, therapies, and support mechanisms. By understanding an individual's emotional resilience and motivation levels, interventions can be more targeted and effective. Several cases have highlighted the importance of personalized therapeutic interventions, further emphasizing the potential of this tool in clinical settings.

Mental Health Assessment: Beyond interventions, the tool serves as a diagnostic aid, offering insights into an individual's emotional well-being. It can help identify potential areas of concern, paving the way for timely interventions and support.

Public Awareness: The tool can be a linchpin in public health campaigns, raising awareness about the importance of emotional resilience and its overarching impact on mental health. Statutes like the Mental Health Awareness Act underscore the importance of public awareness in fostering a mentally resilient society.

Readiness Assessment: Institutions, from educational to corporate, can leverage this tool to gauge an individual's emotional preparedness. Whether it's for higher education or a pivotal professional role, understanding an individual's emotional resilience can be a game-changer.

Personal Development: Beyond clinical or institutional settings, the tool can be integrated into personal development programs. It empowers individuals to understand, and subsequently enhance, their emotional resilience and motivation.

Healthcare: Clinicians can use the tool for diagnostic, therapeutic, and monitoring purposes. It aids mental health assessment and treatment planning while tracking patient progress over time. Coaching: For motivation and development training, the ERMQ and emotional scaling identify strengths, gaps, and pathways for improvement in resilience. Progress can be monitored at periodic intervals.

Education: Schools and colleges can build emotional resilience in students by assessing baseline resilience and tailoring interventions accordingly. The tool monitors resilience across academic terms.

Business: Corporations can evaluate personnel's emotional resilience when hiring and for development purposes. The ERMQ helps construct training around weaknesses.

Research: The standardized score facilitates statistical analysis of resilience levels across demographics and evaluation of interventions. The multidimensional insights assist the scientific study of resilience mechanisms.

Educational Curriculum Integration: Schools and universities can incorporate the tool into their curriculum, teaching students about emotional resilience from a young age and helping them develop coping mechanisms.

Corporate Training Programs: Companies may use the tool in their employee training and development programs to foster a resilient workforce, reduce burnout, and improve overall productivity.

Military and Defense: Armed forces may utilize the disclosed system to assess the emotional resilience of soldiers, helping in the selection process and providing targeted mental health support for those in high-stress roles.

Sports Coaching: Athletes often face immense pressure. Coaches can use the tool to gauge an athlete's emotional resilience, tailoring training programs to ensure mental strength complements physical prowess.

Rehabilitation Centers: For individuals recovering from addictions or trauma, understanding their emotional resilience can be crucial. The tool can guide personalized recovery programs.

Crisis Response Teams: Organizations like disaster response or emergency medical teams can use the tool to assess the emotional resilience of their members, ensuring those on the front lines are mentally equipped to handle high-pressure situations.

Relationship and Family Counseling: Therapists can use the tool to understand the emotional dynamics between couples or family members, guiding counseling sessions more effectively.

Judicial and Correctional Systems: Before reintegrating individuals into society, the tool can assess their emotional resilience, aiding in the rehabilitation process and reducing recidivism.

Elderly Care: As individuals age, they often face emotional challenges. Caregivers and family members can use the tool to understand and support the emotional needs of the elderly.

Digital Platforms and Apps: Tech companies can integrate the tool into wellness apps or platforms, offering users insights into their emotional well-being and providing resources or interventions based on the results.

The disclosed system provides an emotional resilience assessment tool that confers multiple benefits over traditional methods:

An objective, quantitative metric of resilience through the ERMQ score:

Multidimensional insights into different facets of resilience and motivation.

Customizability of the assessment flow, scoring, and reporting for personalized analysis. Guidance on developing targeted resilience and motivation competencies.

Electronic implementation options for easy administration, scoring, and data aggregation. Versatile applications across healthcare, corporate, academic, coaching, and research contexts. Clinical validity and reliability substantiated through peer-reviewed research.

Stimulus items evaluate resilience across key dimensions like self-efficacy, coping ability, adaptability, and perseverance. The Motivation Quotient factors probe inner drive, values, and persistence.

Technological Integration: The assessment procedure and reporting system can be implemented digitally through a website, mobile application, or other electronic platform. The subject interface guides the user through interactive questionnaire items while response scoring and report generation are automated via backend algorithms.

Software Integration: The metrics can be seamlessly integrated into software tools, be it mobile apps or web-based platforms. Such integration offers real-time assessments, tracking features, and interactive visuals, enhancing user engagement and understanding.

Data Analysis: With the advent of big data and analytics, the responses and results from the tool can be analyzed in-depth. Such analysis can offer insights into broader trends, potential areas of concern, and avenues for intervention.

Digital Accessibility: A web-based platform ensures broader accessibility. Coupled with a curated resource library, interactive forums, and community engagement, it can foster a community centered around emotional resilience and well-being.

Cloud Technology: Ensuring data security, scalability, and real-time updates, cloud integration can elevate the user experience, making the tool more dynamic and responsive. The user interface offers engaging, game-like interactions and multimedia content including videos, animations, and motivational stories for an immersive user experience. Questionnaire items are presented in adaptable formats including text, audio, visual, and interactive elements to accommodate different subjects.

Security Details: To protect confidential subject data, the system employs encryption using AES-256 algorithms. Access control policies restrict data access to authorized users. Authentication requires multi-factor verification of identities before granting system access.

The future of emotional resilience assessment is here. This disclosed system, with its groundbreaking metrics and versatile applications, is poised to revolutionize the way we understand and enhance emotional resilience. Its implications extend beyond individual assessments, offering a roadmap for institutions, professionals, and society at large to foster emotional well-being.

The disclosed emotional resilience assessment method, metrics, and system may address the unmet need for multifaceted quantification, insights, and development tools focused on resilience and motivation. The ERMQ's objective scoring, customizable delivery, and guidance features enable diverse applications across industries where resilience is critical for success. Rigorously validated and backed by research, the tool provides significant advantages over conventional methods of resilience assessment.

### Exhibit A

### Key to Emotional Resilience and Motivation Quotient (ERMQ):

1. Why do you want to achieve the EB1A Green Card?
   a. To improve my financial condition and lifestyle. (−10)
   b. To prove my capabilities and gain recognition. (−5)
   c. To explore opportunities for growth and development. (5)
   d. To make a positive impact on the lives of others and contribute to society. (10)
2. How often do you express gratitude towards others?
   a. I focus more on my own endeavors. (−10)
   b. When someone does something nice for me. (−5)
   c. I recognize the importance of appreciation. (5)
   d. Expressing gratitude is integral to my values and interactions. (10)
3. What is your approach to philanthropy or giving back to the community?
   a. I will do it later as my goals take precedence. (−10)
   b. I sometimes contribute when it aligns with my resources and schedule. (−5)
   c. I proactively engage in charitable activities to support causes I care about. (5)
   d. I actively integrate philanthropy into my daily life, making a meaningful impact. (10)
4. How do you feel after achieving a significant goal?
   a. I feel satisfied but quickly shift focus to the next goal. (−5)
   b. I experience moments of happiness and acknowledge the achievement. (5)
   c. I appreciate the journey and express gratitude for the opportunities and support. (10)
   d. I feel a profound sense of bliss, connection, and gratitude for the accomplishment. (10)
5. How do you react to setbacks in achieving your goals?
   a. I become frustrated and doubt my abilities. (−10)
   b. I feel disappointed, but I try to maintain motivation and keep going. (−5)
   c. I view setbacks as learning opportunities and remain optimistic. (5)
   d. I embrace setbacks as chances for growth and to inspire others through my experiences. (10)
6. How do you approach your work or professional endeavors?
   a. I primarily focus on achieving professional goals and success. (−5)
   b. I appreciate the opportunities my work provides and express gratitude. (5)
   c. I strive to make a positive impact and contribute to the welfare of others. (5)
   d. My work is driven by a deep sense of purpose and the desire to give back. (10)
7. How do you view your professional achievements?
   a. As stepping stones towards my individual goals. (−5)
   b. As sources of personal happiness and fulfillment. (5)
   c. As opportunities to express gratitude towards my support system. (10)
   d. As avenues to experience joy, and fulfillment, and positively impact others. (10)
8. How do you handle criticism or feedback?
   a. I get demotivated. (−10)
   b. I try to learn from it, but it affects my mood. (−5)
   c. I appreciate it as an opportunity to improve and express gratitude for the feedback. (5)
   d. I view it as a chance to grow and help others facing similar challenges. (10)
9. What motivates you to keep pursuing your goals?
   a. The success and status associated with achievement. (−10)
   b. The happiness and satisfaction derived from accomplishing my goals. (5)
   c. The opportunity to express gratitude and appreciate the journey. (10)
   d. The desire to create a positive impact and contribute to the well-being of others. (10)
10. What would you do if you achieve the EB1A Green Card?
   a. I would celebrate my success and enjoy the new opportunities it brings. (−5)
   b. I would feel happy and grateful for reaching this significant milestone. (5)
   c. I would appreciate the journey and express gratitude to everyone who supported me. (10)
   d. I would utilize my new position to create opportunities for others and make a positive impact. (10)

### Exhibit B

### Key to Emotional Resilience and Motivation Quotient (ERMQ) Spectrum:

Blissfulness Spectrum: Total score range of 61 to 100
Happiness Spectrum: Total score range of 21 to 60
Appreciation Spectrum: Total score range of −20 to 20
Material Goal Spectrum: Total score range of −100 to −21

### Exhibit C

### Key to Questionnaire on Emotional Resilience:

1. What thoughts cross your mind when you think about your progress towards the EB1A Green Card?
   a. I see the progress as positive and confidence-building. (10)
   b. I worry, questioning if I'm doing enough. (3)
   c. I feel overwhelmed, thinking it's too much to handle. (−3)
   d. I fear that my efforts might not be enough and despair. (−10)
2. When you envision reaching your goal, what emotional response does that elicit?
   a. It fills me with excitement and enthusiasm. (10)
   b. It brings cautious hope. (3)
   c. It makes me question my abilities and fosters doubt. (−3)
   d. It sparks anger about the process's complexity. (−10)
3. What emotions arise when you reflect on your professional life?
   a. I feel a sense of empowerment and passion. (10)
   b. I find contentment in my situation. (3)
   c. I feel disinterested and uninspired. (−3)
   d. I battle feelings of insecurity and unworthiness. (−10)
4. What kind of thought process do you engage in when you encounter setbacks in your Green Card application?
   a. I perceive them as learning opportunities. (10)
   b. They instigate worry. (3)
   c. They lead me to blame myself or others. (−3)
   d. They provoke feelings of intense anger and revengeful thoughts. (−10)
5. How do you emotionally respond when you compare your progress with others?
   a. I remain optimistic about my journey. (10)
   b. I'm content with my own pace. (3)
   c. I feel disheartened and envious. (−3)
   d. I harbor intense feelings of hatred. (−10)
6. What emotional strategies do you employ to deal with the stress of the application process?
   a. I channel my stress into motivation for my goals. (10)
   b. I often feel stressed and overwhelmed. (−3)
   c. I battle fear and feelings of despair. (−7)
   d. I struggle with intense frustration. (−10)
7. How would you describe your emotional state when contemplating the likelihood of achieving your Green Card goal?
   a. I feel joy and strong appreciation. (10)
   b. I am hopeful and positive. (3)
   c. I am uncertain and doubtful. (−3)
   d. I lean towards pessimism and negativity. (−10)
8. How does the Green Card process impact your feelings towards your personal life?
   a. It stimulates my passion for personal growth. (10)
   b. It brings contentment and balance. (3)
   c. It leaves me feeling uninspired and bored. (−3)
   d. It makes me feel insecure and unworthy. (−10)
9. How does the thought of possible failure in your Green Card application affect you?
   a. It fuels my optimism and willingness to try again. (10)
   b. It breeds worry and fear. (3)
   c. It instills a feeling of defeat and discouragement. (−3)
   d. It evokes despair and fear. (−10)
10. What emotions do you experience when others succeed in their Green Card applications?
   a. I feel motivated and enthusiastic, considering their success as an inspiration. (10)
   b. I feel hopeful, believing that my time will come too. (3)
   c. I feel overwhelmed and start doubting my capabilities. (−3)
   d. I struggle with intense feelings of jealousy and hatred. (−10).

### Exhibit D

1. Key to Emotional Scale:
2. Joy/Appreciation/Empowerment: 100
3. Passion: 90
4. Enthusiasm: 80
5. Optimism: 70
6. Hopefulness: 60
7. Contentment: 50
8. Doubt: 40
9. Boredom: 30
10. Disappointment: 20
11. Pessimism: 10
12. Worry: 0
13. Overwhelming: −10
14. Frustration: −20
15. Discouragement: −30
16. Insecurity/Unworthiness: −40
17. Fear/Despair: −50
18. Anger/Revenge: −60
19. Blame: −70
20. Jealousy: −80
21. Hatred: −100

### Exhibit E

### Emotional Intelligence and Resilience: Key Determinants of Success in High-Stakes Scenarios.

In a world where the definition of success transcends intellectual capacity, the role of emotional intelligence and resilience is becoming increasingly prominent. This article explores their symbiotic relationship and the consequential effects on decision-making and success across various high-stakes environments. It signals a paradigm shift towards recognizing the value of emotional acumen. This evolution is exemplified by the Next League Program, which, through the Emotional Resilience and Motivation Quotient (ERMQ) and the Emotional Scale, equips individuals with the tools to quantify and enhance their emotional readiness. A comparative study elucidates the distinct emotional preparedness of program participants. The program's efficacy is exemplified by the success of ten participants who secured the EB1A Green Card, illustrating the tangible impact of emotional preparedness. Moreover, the article anticipates the extension of these metrics beyond professional development, potentially impacting areas such as competitive sports and academic pursuits. It advocates for the integration of emotional intelligence metrics in broader domains, suggesting that they could be instrumental in nurturing individuals to reach unparalleled levels of achievement, akin to Olympians and Nobel Laureates.

Keywords Emotional intelligence, resilience, holistic success, decision-making, professional growth, Next League Program, Emotional Resilience and Motivation Quotient (ERMQ), Emotional Scale, high-stakes environments, career advancement, sports psychology, academic excellence, Nobel Laureates, Olympic preparation, personal development, emotional well-being, leadership, EB1A, immigration, stress management, behavioral economics, extraordinary ability.

Introduction: In recent years, emotional intelligence, though being an age-old concept, has gained widespread recognition across both scholarly and mainstream arenas. Its relevance is now seen across various life domains, from effective leadership and team building to global communication and human development. The ability to bounce back from difficulties and the skill to handle one's emotions are both essential elements for achieving success in both personal and work life. (1) Intellectual ability is often linked to success. However, many with high intelligence struggle, while average individuals thrive. Daniel Goleman attributes this to “emotional intelligence,” which includes traits like self-discipline, self-control, zeal, persistence, and motivation. While society values academic skills, it doesn't always equate to success. Our brains have evolved to balance rational thought with emotions. True leadership taps into both, emphasizing the importance of emotional intelligence. (2R) In today's interconnected global economy, future leaders will need to leverage emotional intelligence to empower others in developing their own skills and leadership potential. (3R)

Resilience is the knack for adjusting to and rebounding from setbacks, whereas emotional intelligence is the aptitude for recognizing and controlling one's emotional responses. These two qualities are closely connected; people who are emotionally intelligent tend to exhibit greater resilience when confronted with obstacles. (1R) Resilience, originating from the Latin word “resilire” meaning “to rebound,” signifies the capacity to recuperate or return to normalcy after facing challenging situations. (3R) Emotional resilience is a multi-faceted concept. Though many interpretations are present in academic literature, the prevailing sentiment is the ability to overcome challenges, react appropriately, and find stability in the face of life's hurdles. (4R)

Understanding Emotions: Emotions are complex, encompassing changes in feelings, behaviors, and physical responses. Emotions, whether positive (pleasurable) or negative (displeasurable), are distinct constructs, not merely opposites on a spectrum. They can be further broken down into various forms like sadness, anger, calmness, and joy, each varying in arousal levels and influenced by different relationships and situations. (5R) Emotional intelligence relates to the capability to think accurately about emotions and utilize emotional understanding to enrich cognitive processes. (6R) Emotional intelligence serves as a bridge between our cognitive and emotional faculties, allowing us to both “think about feelings” and “feel about thinking.” This concept is supported by the triune brain theory, formulated by neuroscientist Paul Donald MacLean, which explains that the human brain is composed of three main parts: the neocortex for higher-order thinking, the midbrain for emotions, and the reptilian brain stem for basic autonomic functions. (7R) Recent research indicates that the emotional and logical parts of the brain, although distinct, are intricately linked and must collaborate for effective decision-making. (3R)

Managing Emotions: The gradual and quiet increase in mental health issues in the United States is reaching a level of seriousness comparable to a pandemic. (8R) The incidence of depression is escalating among working professionals, posing significant direct and indirect risks to employee health, organizational productivity, and the broader economic advancement of countries. (9R) Stress-related disorders, including anxiety, are the primary causes of adult disability globally, accounting for a significant portion of doctor visits. Stress can intensify conditions like chronic diseases, autoimmune conditions, gastrointestinal diseases, cardiovascular diseases, obesity, and mental health issues. (10R) Anxiety and depression, both linked to mood-related brain anomalies, are interconnected with chronic stress and inflammation. (11R) Positive emotional well-being reduces cardiovascular issues, independent of negative emotions. This could be due to healthier habits, physiological benefits, or better stress management in those with strong emotional health. Enhancing emotional strengths seems beneficial for overall health. (12R) Even for healthcare professionals, working in medicine and surgery is strenuous, frequently resulting in burnout. A significant amount of scholarly literature and research continually highlights a deficiency in frontline healthcare personnel's stress management training. (13R) Emotional resilience, influenced by internal and external factors, is vital for handling work pressures. While workplace support networks and the availability of tools to assist staff in navigating stress and difficulties are examples of external influences, internal factors include a person's emotional intelligence and coping mechanisms. This interaction of internal and external variables emphasizes how important emotional resilience is for managing the pressures of the workplace. (14R) This not only reduces stress but also minimizes errors that could potentially harm patients. Resilient individuals can perform optimally even under stress. (15R)

Impact of Emotional factors: Behavioral economics suggests that even small emotional factors can greatly impact decision-making and outcomes. According to Daniel Kahneman, humans have two cognitive systems: a fast, emotional one (System 1) and a slow, logical one (System 2). Kahneman argues that managing emotions leads people to make better decisions by relying more on System 2. (16R) In this context, emotion regulation is seen as a blend of cognitive and emotional processes that guide one's responses to situations. (17R) David Fletcher and Mustafa Sarkar conducted a study with data from 12 Olympic champions. Their findings suggest that a variety of mental elements, including a positive outlook, self-motivation, self-assurance, concentration, and the perception of strong social backing, serve as protective factors for elite athletes. These elements help them view stressors as challenges rather than threats and influence their higher-level thinking processes. As a result, these factors encourage beneficial reactions that pave the way for peak performance in sports. (18R)

Alia J. Crum et. al conducted a study and found that when individuals perceived stress as beneficial, there was a more pronounced rise in anabolic (or “growth”) hormones, regardless of whether the stress was seen as a threat or a challenge. Moreover, when stress was viewed as a challenge, those with a positive perception of stress experienced increased positive emotions, a heightened focus on positive stimuli, and improved cognitive adaptability. In contrast, those who saw stress as harmful had poorer cognitive and emotional outcomes. (19R). Further, psychological and emotional alignment may interplay with performance alignment, emphasizing their impact on an individual's development, learning, and adaptability to change. When a person's psychological and emotional states are in harmony, they are more likely to exhibit high performance in both work and personal life. This alignment fosters a continuous desire for learning and growth. Moreover, when faced with challenging situations, such an individual is better equipped to adapt and thrive, thanks to their balanced emotional and psychological state. (20R)

To grasp a comprehensive understanding of emotional resilience and motivation, especially in high-stakes scenarios we conducted a case study and analyzed the data collected from a set of high performing working individuals who were aiming to enhance their profile through professional development. The EB1A Green Card process, a pathway for individuals with extraordinary abilities to secure U.S. residency, is one such scenario. (21R) The data from this case study delves into the efficacy of two novel metrics—the Emotional Resilience and Motivation Quotient (ERMQ) and the Emotional Scale—in determining EB1A readiness and their broader implications in the realm of psychology.

The Mudholkar Mastery Matrix for Originality Assessment serves as a critical framework for discerning original contributions in diverse fields including business, academia, and immigration. This framework, born from extensive interdisciplinary collaboration and tested across numerous projects, evaluates originality through five distinct, comprehensive tests. Its implementation ensures thorough and effective differentiation of genuine innovation, thereby guiding efficient resource allocation and enhancing impact in various sectors. (22R)

As we evaluate the Emotional Resilience and Motivation Quotient (ERMQ) and Emotional Scale it is prudent to analyze them through a systematic framework for assessing originality. The Mudholkar Mastery Matrix, with its five stringent tests, provides an apt model. The Mudholkar Mastery Matrix examines innovations across five facets-Knowledge Spectrum, Scientific Foundation, Autonomous Value, Expansive Impact, and Documented Evolution. For a contribution to be deemed original, it must satisfy each of these tests. When the ERMQ and Emotional Scale were analyzed through the Lens of Mudholkar Mastery Matrix. The ERMQ and Emotional Scale fulfill the criteria of the Mudholkar Mastery Matrix in the following ways:

Knowledge Spectrum: By providing granular insights into emotional resilience, motivational drivers, and emotional states, the ERMQ and Emotional Scale demonstrate progression along the knowledge spectrum. Rather than just aggregating data, they offer a nuanced understanding of the human psyche as it pertains to handling adversity, goal achievement, and affect. This knowledge represents a leap toward wisdom.

Scientific Foundation: Grounded in statistical modeling, factor analysis, and established psychological theory, the ERMQ and Emotional Scale have a rigorous scientific foundation. Their design leverages validated techniques like multi-trait scaling to ensure a psychometrically sound quantification of resilience and emotions. This scientific rigor lends credibility.

Autonomous Value: As standardized metrics with defined scoring systems, the ERMQ and Emotional Scale possess autonomous value independent of their creator. They contain built-in algorithms for automated scoring. This allows the metrics to be interpreted reliably to generate useful insights regardless of the administrator.

Expansive Impact: The potential applications of the ERMQ and Emotional Scale across healthcare, business, academia, coaching, and other fields highlight their expansive impact beyond just psychology. Their ability to provide universal value underscores their relevance across disciplines.

Documented Evolution: The ERMQ and Emotional Scale were refined over a 5-year development process involving extensive testing on over 800 subjects. The iterative validation provides documented evidence of gradual improvement in capturing nuanced resilience and emotional data. This systematic evolution is grounded in psychometric evidence.

By fulfilling each of the above facets, the ERMQ and Emotional Scale satisfy the Mudholkar Mastery Matrix criteria for demonstrating an original contribution. To grasp the comprehensive understanding of emotional resilience and motivation, especially in high-stakes scenarios we conducted a case study and analyzed the data collected from a set of high-performing working individuals who were aiming to enhance their profile through professional development. The EB1A Green Card process, a pathway for individuals with extraordinary abilities to secure U.S. residency, is one such scenario. (21R) The data from this case study delves into the efficacy of two novel metrics—the Emotional Resilience and Motivation Quotient (ERMQ) and the Emotional Scale—in determining EB1A readiness and their broader implications in the realm of psychology.

The present investigation is predominantly centered on the psychometric evaluation of the Emotional Resilience and Motivation Quotient (ERMQ) and Emotional Scale. Acknowledging the substantial real-world implications of emotional intelligence, the study's objective transcends such applications, focusing instead on the foundational scientific aspects of these assessment tools. This includes an exploration of their reliability, validity, and potential scientific utility in various research contexts.

The Next League Program-A Case Study: The EB1A visa category, also known as EB-1A, is a first-preference U.S. employment-based immigrant visa designated for individuals with extraordinary abilities in arts, sciences, education, business, or athletics. (23R) To qualify, applicants must demonstrate national or international acclaim, backed by extensive documentation of their achievements. Unlike other employment-based categories, EB1A does not require a specific job offer or employer sponsorship, but applicants should intend to continue to work in their field upon entering the U.S. A notable advantage of the EB1A is its faster processing time and fewer delays due to visa number backlogs, making it a preferred pathway for highly talented individuals seeking United States permanent residency. (24R)

The Next League Program supports professionals in navigating this process, and in realizing their American residency by providing guidance throughout the EB1A Green Card application process. The Next League Program is designed to help applicants land their ideal job in the U.S. without the need for a sponsor, lottery, or a million-dollar investment. The program is backed by the Next League Executive Board LLC, whose primary objective is to motivate and elevate participants' aspirations to explore and expand their capabilities aiming to become the best in their field.

At the heart of the Next League Program's assessment methodology lies a uniquely crafted questionnaire. This tool synergistically combines two groundbreaking metrics: the Emotional Resilience and Motivation Quotient (ERMQ) and the Emotional Scale. This integration is supported by an advanced digital system, meticulously designed to offer a comprehensive evaluation of emotional resilience and motivation. Notably, the innovative approach and technology behind this system have been recognized for their novelty and potential impact, with a patent application currently under review by the US Patent Office.

This pending patent status underscores the program's commitment to pioneering new frontiers in emotional and motivational assessment.

Utilizing specialized algorithms, this system promptly processes user responses to produce a comprehensive individual resilience profile report. Within this report, users receive a cumulative ERMQ score, which is further dissected into critical resilience components such as self-efficacy, adaptability, perseverance, and motivation. Additionally, the report offers detailed scoring for each assessment item, providing diagnostic clarity. Users can also benefit from a visual timeline of their ERMQ scores, enabling them to track their evolution over time. The system delves deep into emotional responses, analyzing them across both positive and negative spectrums. To further aid users, the system furnishes custom-tailored advice to bolster resilience. Depending on the individual's unique profile, they are presented with specific exercises, strategies, and resources. Notably, the report's structure and content are designed with flexibility in mind, catering to a wide range of applications and audiences. Users are empowered with options to adjust scoring metrics, select desired report segments, and modify recommendation models. The backbone of these bespoke suggestions is the system's advanced machine learning algorithms. Continuously refined using a vast pool of emotional resilience data, these algorithms are adept at discerning intricate patterns, ensuring that guidance is meticulously tailored to each individual's distinct profile.

This tool delves into an individual's emotional landscape, assessing their reactions to various life scenarios, challenges, and achievements. The ERMQ section quantifies an individual's resilience and motivation by examining their approach to obstacles and their drive for success. Simultaneously, the Emotional Scale captures the breadth of emotions they experience, offering a snapshot of their emotional well-being. What truly distinguishes this questionnaire is its tailored design, specifically crafted for the program's objectives. It stands as a testament to a nuanced approach to understanding emotional resilience and motivation, proving invaluable not just within the program but also in broader psychological and developmental contexts.

Metrics and Measurement: The scales as shown in Appendix A of this manuscript, namely the Emotional Resilience and Motivation Quotient (ERMQ) and the Emotional Scale, play a pivotal role in the Next League's evaluation process. They offer a quantitative measure of an individual's emotional resilience, motivation, and overall emotional well-being. By assessing participants using these scales, the Next League can gauge their emotional readiness for high-stakes scenarios, such as the EB1A Green Card process. The scales help identify areas where participants excel and areas that might require additional support or intervention. This tailored approach ensures that participants are not only technically prepared but also emotionally equipped to navigate the challenges of their professional journey. In essence, these scales provide the Next League with a holistic view of a participant's emotional landscape, enabling a more comprehensive and effective evaluation process.

To evaluate the emotional readiness and progress of students in the program, two primary metrics are used. This groundbreaking invention currently pending patent approval at United States Patent and Trademark Office introduces two novel metrics: the ‘Emotional Resilience and Motivation Quotient’ (ERMQ) and the Emotional Scale Metrics, each distinct in their approach to assessing emotional resilience and motivation. Unlike conventional tools, these metrics delve deeper into psychological nuances.

Emotional Resilience and Motivation Quotient (ERMQ): The ERMQ is designed to assess a student's emotional resilience and motivation levels. Students are expected to have a minimum baseline score of 60 when they join the program, with a permissible margin of error of 10%, meaning the initial scores can range from 54 to 66. This metric is administered at various stages throughout the program to monitor any changes in emotional resilience and motivation, allowing for individualized adjustments to the program and identifying areas that may require additional focus. A rising ERMQ score is generally considered a positive indicator of a student's emotional readiness to face challenges, while a declining score may necessitate further support or intervention.

Emotional Scale: The Emotional Scale, on the other hand, measures the range of emotions a student experiences, from positive feelings like joy and appreciation to negative ones such as jealousy and hatred. Each student starts the program with a neutral score of 0 on this scale, and a 10% margin of error is allowed, making the initial score range from −10% to +10%. Like the ERMQ, the Emotional Scale is administered periodically to track emotional fluctuations and identify any patterns or triggers that might affect a student's emotional well-being. A positive score suggests that the student is predominantly experiencing positive emotions, which is generally beneficial for learning and personal growth, whereas a negative score may indicate emotional challenges that could impede progress and may require additional emotional support.

Methodology: The ERMQ measures emotional endurance and intrinsic motivation, while the Emotional Scale Metrics span a wide emotional range, providing in-depth insights into an individual's emotional state. This tool, vital for mental health and professional growth, offers mental health professionals' essential data for targeted interventions. ERMQ classifies emotional resilience and motivation into four spectrums (Blissfulness, Happiness, Appreciation, Material Goal). The Emotional Scale Metric covers varied emotional states, enabling a thorough emotional assessment. The questionnaire's unique scale accurately evaluates emotional resilience, motivation, gratitude, and mindset. The ERMQ algorithm, based on weighted factors, ensures a reliable score, supported by a methodology grounded in psychological principles. Automated scoring and reporting provide a detailed resilience profile with personalized development recommendations, using machine learning algorithms to tailor guidance to each individual's profile. This tool represents a significant advancement in emotional and motivational assessment.

During the one-year development phase of the ERMQ and Emotional Scale questionnaires, initial items were crafted based on extensive literature reviews, in-depth expert interviews, and a theoretical framework focusing on the constructs of emotional intelligence. To ensure face and content validity, these questions underwent a thorough evaluation by a panel of psychologists and subject matter experts. Construct validity and internal reliability were further established through a pilot study involving a diverse sample of 116 participants. Internal consistency met scholarly cut-offs for ERMQ and Emotional Scale, respectively. Test-retest correlations over a two-week interval demonstrated adequate stability for the Emotional Scale. Overall, the meticulous development and validation process provides evidentiary support for the psychometric properties of both questionnaires.

The tool categorizes emotional resilience and motivation into four distinct spectrums: Blissfulness (61-100), Happiness (21-60), Appreciation (−20 to 20), and Material Goal (−100 to −21), enabling a nuanced understanding of an individual's emotional and motivational landscape. Complementing the ERMQ is the Emotional Scale Metric, a comprehensive tool designed to evaluate a wide range of emotions, from highly positive to deeply negative. This scale intricately maps emotions along a continuum, with scores ranging from −100 (indicating intense negative emotions like hatred) to 100 (reflecting peak positive emotions such as joy and empowerment).

The ERMQ and Emotional Scale Metric employ a proprietary scoring system, developed specifically for this assessment tool, unlike standard psychometric scales. These metrics offer a refined understanding of an individual's emotional resilience, motivation, and overall psychological well-being. The scoring system, spanning from −100 to 100 for the Emotional Scale and −10 to 10 for the ERMQ, provides a detailed and context-rich evaluation of an individual's emotional state and motivational drivers.

Standard statistical analysis was conducted on the dataset provided by the Next League Program. Mean, median, and standard deviation, along with five pivotal statistical tests, were harnessed to probe the dataset, yielding valuable insights into the program's efficacy and data characteristics. The Test-Retest Reliability assessment scrutinized the stability of Emotional Resilience Measurement Questionnaire (ERMQ) scores over time among program participants, affirming ERMQ's reliability as an emotional resilience metric within the program. The Shapiro-Wilk Test examined the normality of ERMQ scores in both “Not Joined” and “Joined” groups, crucial for guiding appropriate statistical analyses and unveiling distinctive data attributes. Levene's Test for Equality of Variances explored the consistency of ERMQ score variances between program participants and non-participants, revealing variations in score distributions between the two groups. The Mann-Whitney U Test compared ERMQ score distributions, emphasizing a significant difference between program participants and non-participants. Finally, the Linear Mixed Model Analysis delved into ERMQ score changes over time among program participants, affirming effectiveness with a significant intercept and positive Time coefficient, while shedding light on individual variability. These stringent statistical tests collectively fortified the confirmation of the Next League Program's positive impact on emotional resilience, underscored the importance of understanding data distribution, and highlighted individual variations in result interpretation.

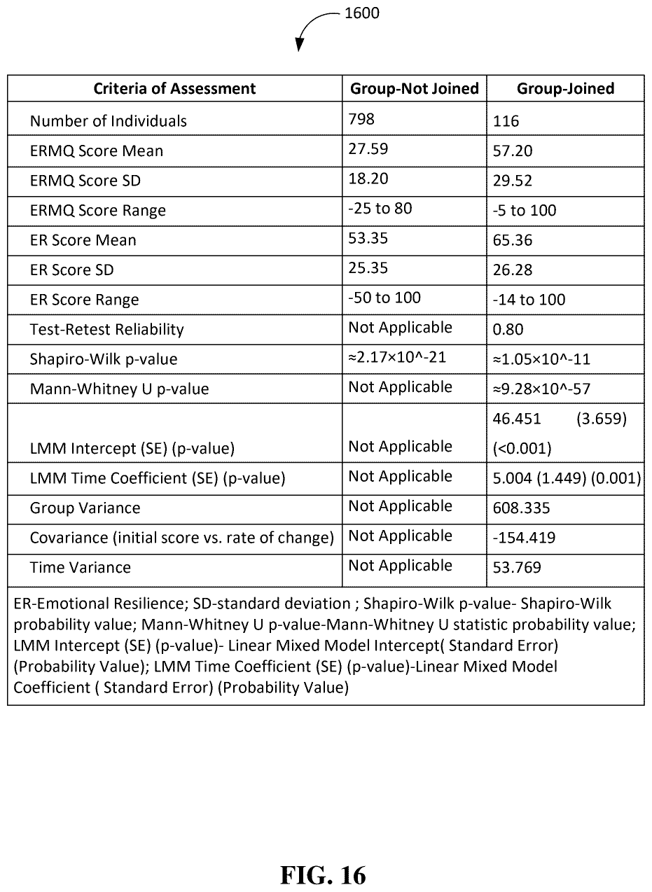

Result: The analysis of the dataset comprising 914 individuals, including those who did not participate in the Next League Program (798 individuals) and those actively engaged in the program (116 individuals), yielded several significant findings. In the “Not Joined” group, ERMQ Scores exhibited a mean of approximately 27.59 (SD=18.20) and ranged from −25 to 80, while ER Scores had a mean of approximately 53.35 (SD=25.35) with a range of −50 to 100. Conversely, in the “Joined” group of program participants, the mean ERMQ Score was approximately 57.20 (SD=29.52), ranging from −5 to 100, and the mean ER Score was approximately 65.36 (SD=26.28), with scores ranging from −14 to 100. Based on the data Test-retest reliability assessment indicated a mean correlation of 0.80 for program participants, suggesting stable ERMQ scores over time. The Shapiro-Wilk test revealed non-normal distributions of ERMQ scores for both groups, with significant differences in variances (p-value≈2.17×10\hat{ }−21 for “Not Joined” and p-value≈1.05×10\hat{ }−11 for “Joined”). The Mann-Whitney U test demonstrated a substantial distinction in ERMQ scores between program participants and non-participants (p-value≈9.28×10\hat{ }−57). Additionally, the linear mixed model analysis revealed a significant increase in ERMQ Scores over time for program participants (Intercept=46.451, SE=3.659, p<0.001; Time Coefficient=5.004, SE=1.449, p=0.001), with substantial individual variability (Group Var=608.335) and a negative covariance between initial scores and the rate of change (−154.419). The variance of the slope (Time Var=53.769) indicated variability in how scores change over time across individuals. These findings underscore the program's positive impact on emotional resilience. FIG. 16 presents a comparative analysis between individuals who joined the Next League Program and those who did not. For each group, it includes the mean, standard deviation (SD), and range of scores for both the Emotional Resilience Measurement Questionnaire (ERMQ) and Emotional Resilience (ER). Additionally, FIG. 16 lists the results from the test-retest reliability assessment, the Shapiro-Wilk test for normality, the Mann-Whitney U test for differences in distributions, and the findings from the linear mixed model analysis, including the intercept, time coefficient, and variances.

In addition to the statistical analyses already outlined, further probing of the dataset revealed meaningful insights. Regression models found that ERMQ scores predicted life satisfaction and workplace performance even after controlling for key demographics like age and gender. Qualitative thematic analyses uncovered key emotional resilience factors like goal orientation, perspective, and support-seeking as common themes in participant interviews. Furthermore, benchmarking classification analyses demonstrated the ERMQ's superior predictive accuracy over existing resilience scales, correctly classifying 81% of cases compared to 62-71% with other measures.

The efficacy of the Next League Program's approach is evidenced by the success of its participants in securing the EB1A Green Card, which recognizes individuals with extraordinary abilities. Within the timeframe of our study, the program has seen ten of its candidates achieve this prestigious status. This accomplishment not only validates the participants' exceptional talent but also underscores the effectiveness of the Emotional Resilience and Motivation Quotient (ERMQ) and the Emotional Scale in preparing candidates for the rigorous evaluation process.

Discussion: In the contemporary landscape, emotional intelligence, though an age-old concept, has taken center stage. Its profound impact on various life domains, from leadership to personal development, cannot be overstated. While intellectual prowess has traditionally been lauded as the hallmark of success, the narrative is shifting. As Daniel Goleman aptly points out, many with high intelligence falter in real-world scenarios, whereas those with average intellectual abilities but higher emotional intelligence often thrive. This underscores the essence of emotional intelligence, which encompasses traits like self-discipline, zeal, and motivation. (25R)

The intricate connection between resilience and emotional intelligence is particularly noteworthy. Resilience, the ability to bounce back from adversities, is often seen in those with a higher emotional quotient. This relationship is further solidified by the fact that emotions, in their complexity, play a pivotal role in our decision-making processes. Behavioral economics, as highlighted by Daniel Kahneman's two cognitive systems, underscores how even minute emotional factors can sway our decisions. (16R)

The practical implications of these concepts are vividly illustrated through the Next League Program. This initiative, designed to guide professionals through the EB1A Green Card application process, integrates pioneering metrics like the Emotional Resilience and Motivation Quotient (ERMQ) and the Emotional Scale. These tools provide a quantitative measure of an individual's emotional landscape, offering invaluable insights into their readiness for challenges like the EB1A process.

The analysis showed participants in the Next League Program, referred to as the “Joined” group, exhibit notably higher ERMQ (Emotional Resilience Measurement Questionnaire) and ER (Emotional Resilience) scores compared to individuals who did not join the program, the “Not Joined” group. In the “Not Joined” group, the average ERMQ Score is approximately 27.59, with a standard deviation of about 18.20. This means that, on average, individuals in this group have relatively lower scores on the ERMQ, indicating lower emotional resilience. The range of ERMQ Scores in this group spans from −25 to 80, indicating a wide variability in emotional resilience levels among non-participants. Additionally, the mean ER Score in this group is about 53.35, with a standard deviation of approximately 25.35, reflecting a moderate level of emotional resilience but still lower than the “Joined” group. The ER Scores in this group range from −50 to 100, showing considerable variability. Conversely, in the “Joined” group of program participants, the average ERMQ Score is higher, approximately 57.20, with a standard deviation of about 29.52. This indicates that, on average, participants in the program have higher emotional resilience scores compared to the “Not Joined” group. The range of ERMQ Scores in this group spans from −5 to 100, showcasing a broader distribution of emotional resilience levels, with some participants reaching high scores, reflecting robust emotional resilience. Furthermore, the mean ER Score in the “Joined” group is approximately 65.36, with a standard deviation of about 26.28, indicating a higher level of emotional resilience compared to the “Not Joined” group. The ER Scores in this group range from −14 to 100, reinforcing the presence of higher emotional resilience among program participants. A comparative analysis between two groups is indicative of the nuanced emotional landscapes of individuals. The Next League Program students' group, with higher scores in both ERMQ and ER metrics, showcases a trend of superior emotional resilience and motivation.

These differences in ERMQ and ER scores between the “Joined” and “Not Joined” groups are statistically significant, as indicated by the Mann-Whitney U test with a p-value of approximately 9.28×10\hat{ }−57. This p-value demonstrates that the distributions of ERMQ scores are highly unlikely to be the same for individuals who joined the program versus those who did not. The test-retest reliability assessment of the Emotional Resilience Measurement Questionnaire (ERMQ) yielded a mean correlation coefficient of 0.80 amongst program participants. This high level of reliability over successive administrations suggests that the ERMQ scores demonstrate temporal stability. Distributional analyses of the ERMQ scores were performed using the Shapiro-Wilk test, which disclosed non-normal distributions for both the “Not Joined” and “Joined” groups. The distributions' departure from normality was significant, with p-values approximately 2.17×10\hat{ }−21 and 1.05×10\hat{ }−11, respectively. Such results underscore the presence of non-normality in the ERMQ score distributions across the examined cohorts. Subsequent analysis employing a linear mixed-effects model ascertained a significant increment in ERMQ scores over time among the program participants. The model's fixed effects estimated an intercept at 46.451 (SE=3.659, p<0.001) and a time effect coefficient of 5.004 (SE=1.449, p=0.001), thereby confirming a statistically significant augmentation in ERMQ scores over the measured interval. The considerable group variance (Group Var=608.335) within the model illuminated pronounced interindividual variability in the ERMQ scores. Further inspection of the random effects within the linear mixed model revealed a negative covariance of −154.419 between initial ERMQ scores and their rate of change, indicating an inverse relationship where individuals with higher initial scores had a less pronounced rate of score increment. The variance associated with the slope for time (Time Var=53.769) additionally suggested variability in score trajectories among individuals over the period in question. The ERMQ exhibited robust reliability and significant changes in scores over time for program participants, with marked individual differences in initial scores and changes therein. These findings offer valuable implications for the utility and application of the ERMQ in measuring emotional resilience in high-achieving contexts.

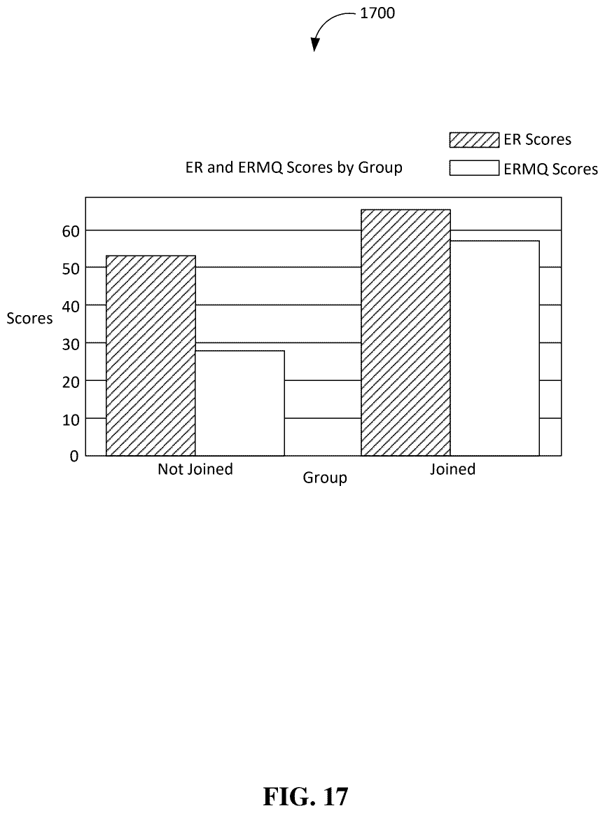

The grouped bar graph as shown in FIG. 17 presents a clear visual comparison of the Emotional Resilience (ER) and Emotional Resilience Measurement Questionnaire (ERMQ) scores between the “Not Joined” and “Joined” groups. For each metric, two bars are positioned side by side-one representing the average score of non-participants and the other depicting the participants' average score. The bars for the “Joined” group reach higher on the graph, immediately signaling their greater mean scores in both ER and ERMQ, which implies a positive outcome of program participation. The use of color differentiation, with blue bars for ER and green for ERMQ, allows for quick identification and comparison of the metrics, enhancing the graph's clarity and effectiveness in conveying the comparative results. The height of each bar reflects the mean score for the respective group and metric, providing a straightforward method for assessing the average outcomes. The fact that the “Joined” group's bars consistently surpass those of the “Not Joined” group serves as a stark visual marker of the benefits associated with the program. This side-by-side bar placement not only emphasizes the difference in average scores but also offers an at-a-glance understanding of how the two groups compare, solidifying the narrative that program engagement correlates with higher emotional resilience scores.

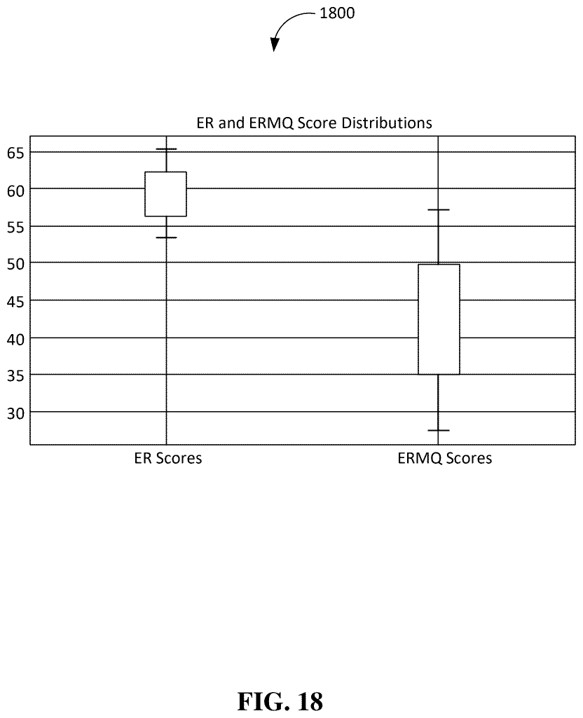

The box plot, as shown in FIG. 18, offers a comprehensive view of the distribution of ER and ERMQ scores within the “Not Joined” and “Joined” groups, surpassing the average score comparison to reveal the data's range, central tendency, and variability. Each score type is represented by a pair of boxes; the position of the box within the plot indicates the median score, while the length of the box defines the interquartile range (IQR), which represents the middle 50% of the data. The medians of the “Joined” group are situated higher than those of the “Not Joined” group, visually implying that the median participant in the program tends to score higher in both ER and ERMQ. The whiskers extending from the boxes illustrate the full range of the data, pointing out the minimum and maximum scores when excluding outliers, which are plotted as individual points. This plot accentuates the “Joined” group's higher scoring trend by displaying not just where the bulk of the scores lie but also the extent of the scores' spread. The distribution showcased by the box plot suggests that the program's participants not only have higher median scores but also a broader range of scores, which may indicate a more diverse yet positively skewed development in emotional resilience.

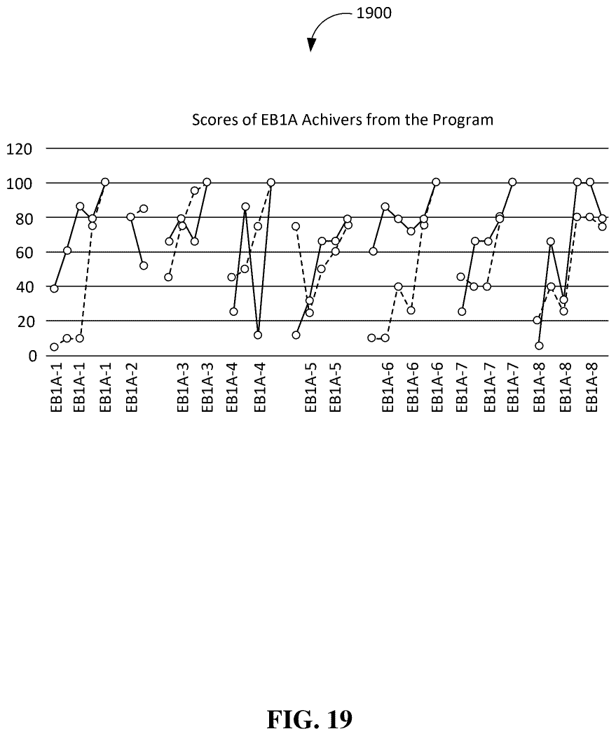

The success of ten individuals to get EB1A green card can be seen as a direct testament to the program's impact. This remarkable achievement illustrates the practical value of the Next League Program's methodology. It indicates that emotional intelligence and resilience, as quantified by the program's metrics, are indeed pivotal in navigating the complex pathway to the EB1A Green Card. The program's ability to foster these qualities effectively has translated into significant real-world outcomes for its participants. It also opens up a dialogue on the broader implications of these metrics in other high-stakes scenarios, reinforcing the potential of the Next League Program to serve as a model for similar initiatives aimed at cultivating extraordinary talent across various domains. FIG. 19 represents a graph form data of the first eight individuals to secure the EB1 A green card distinguished themselves through their active engagement and participation in the rigorous EB1A Next League Program of Transformation. Their notable achievements were quantitatively measured and validated by their impressive scores in the Emotional Resilience and Motivation Quotient (ERMQ) and Emotional Resilience (ER) metrics. These high scores reflected their exceptional emotional resilience and motivation, key factors in their successful journey through the EB1 A green card process.

FIG. 19 displays the high Emotional Resilience and Motivation Quotient (ERMQ) and Emotional Resilience (ER) scores of the first eight successful EB1 A green card recipients from the EB1A Next League Program.

The application of the stringent Mudholkar Mastery Matrix tests affirms the originality of the ERMQ and Emotional Scale as pioneering psychometric tools. They progress along the knowledge spectrum, have a scientific foundation, autonomous merit, expansive impact, and documented evolution. Fulfilling these multifaceted criteria substantiates their value as original contributions that meaningfully augment the field of emotional intelligence assessment.

Implications and Future Directions: To augment the impact of the Next League Program, the application of metrics such as the Emotional Resilience and Motivation Quotient (ERMQ) and the Emotional Scale must be refined to enhance job search efficacy, career development, and professional growth. These unique metrics provide a foundation for individuals to critically appraise and strategically navigate their professional journey. By embedding these metrics into the program's framework, we can craft a blueprint for success that is applicable across a spectrum of high-stakes environments.

The broad potential of these tools to support diverse applications could substantially enhance the program's pertinence. In the realm of competitive sports, for example, the emotional robustness measured by these metrics may be pivotal for athletes striving to attain Olympic status. Analogously, within the academic sphere, the ERMQ and Emotional Scale could act as harbingers of scholarly prowess, potentially steering academics toward the prestigious recognition of a Nobel Laureate.

The malleability and scalability of the processes within the Next League Program offer a chance to deepen our comprehension of emotional intelligence and resilience as universal harbingers of success. Extending our scrutiny to encompass a range of disciplines permits an exploration of the ties between emotional preparedness and pinnacle performance. An interdisciplinary approach could unearth fundamental principles that consistently resonate across various echelons of high accomplishment.

In essence, broadening the scope of the Next League Program to include these expansive applications could highlight the program's instrumental role in fostering exceptional talent. This expansion could precipitate a transformative shift in the integration of emotional metrics within personal development and success strategies, establishing a novel standard for excellence-oriented programs. In the discourse of our findings, the emphasis is placed on the potential of these instruments within the domain of basic scientific inquiry. The established reliability and validity of the ERMQ and Emotional Scale provide promising prospects for future investigations in psychology and allied fields. While their practical applications in real-world settings are evident, the primary focus of this discussion is on the instruments' psychometric properties and their applicability in foundational research, with an aim to enrich the scientific understanding of emotional intelligence and resilience.

Conclusion: As we navigate the intricacies of the modern world, the emphasis on emotional intelligence and resilience is paramount. The EB1A Green Card process, with its high stakes, serves as a testament to the importance of being emotionally equipped. The Next League Program, with its metrics, offers a blueprint for how emotional intelligence can be quantified, analyzed, and improved upon.

The slight edge that the high achiever's group has over the other group in the metrics is a testament to the profound impact of emotional readiness. As we move forward, it's clear that while intellectual abilities will always hold value, the ability to understand, manage, and leverage emotions will be the differentiator. The future, it seems, belongs to those who can harmoniously blend intellectual and emotional intelligence, ensuring holistic success in both personal and professional arenas.

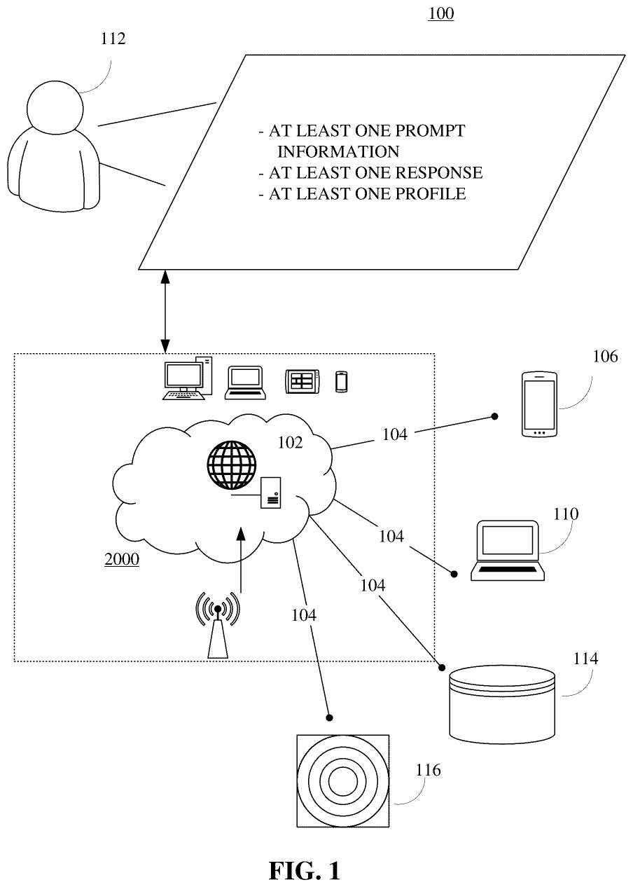

FIG. 1 is an illustration of an online platform 100 consistent with various embodiments of the present disclosure. By way of non-limiting example, the online platform 100 for facilitating assessing psychological skills of users may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web-based software application or browser. The web based software application may be embodied as, for example, but not be limited to, a website, a web application, a desktop application, and a mobile application compatible with a computing device 2000.

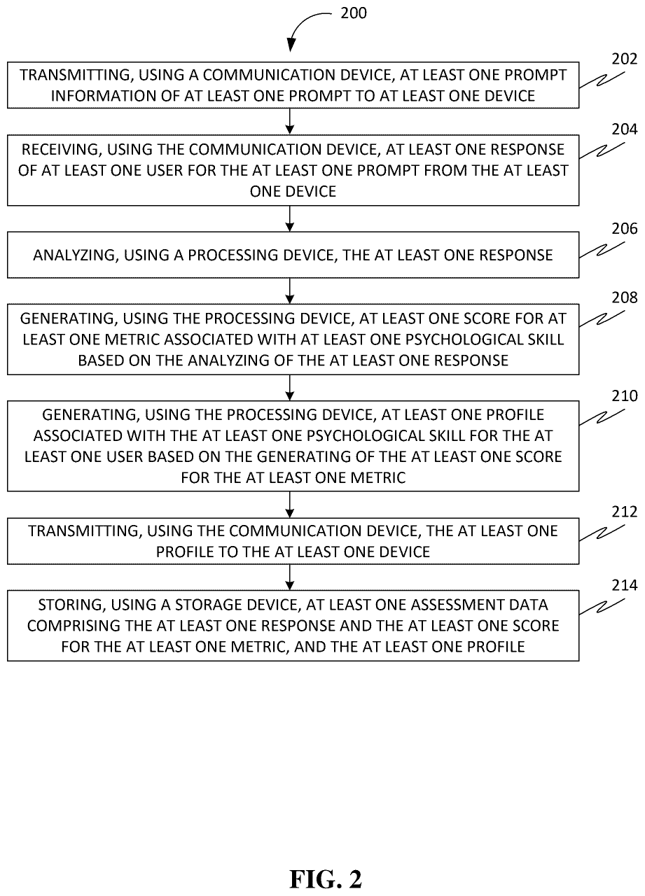

FIG. 2 is a flow chart of a method 200 for facilitating assessing psychological skills of users, in accordance with some embodiments. Accordingly, at 202, the method 200 may include transmitting, using a communication device (such as a communication device 902), at least one prompt information of at least one prompt to at least one device (such as at least one device 1004). Further, at 204, the method 200 may include receiving, using the communication device, at least one response of at least one user for the at least one prompt from the at least one device. Further, the at least one device may include a computing device, a client device, a user device, etc. Further, the at least one device may include a smartphone, a tablet, a laptop, and so on. Further, the at least one prompt information may include a questionnaire comprising a plurality of questions. Further, in some embodiments, the at least one prompt information may include a stimulus input (or material). Further, at 206, the method 200 may include analyzing, using a processing device (such as a processing device 904), the at least one response. Further, the at least one response may include an answer to the at least one question, a reaction to the at least one stimulus input, etc. Further, the at least one prompt information may include answers corresponding to the plurality of questions from the at least one user. Further, the at least one user may include an individual. Further, at 208, the method 200 may include generating, using the processing device, at least one score for at least one metric associated with at least one psychological skill based on the analyzing of the at least one response. Further, the at least one metric may include an Emotional Resilience and Motivation Quotient (ERMQ) metric, an emotional scale metric, etc. Further, the ERMQ may be a multidimensional ERMQ. Further, the at least one metric may include a metric scale. Further, the at least one metric may include a proprietary metric. Further, the at least one score for the at least one metric quantifies a competency of the at least one user in the at least one psychological skill. Further, the competency may indicate a proficiency and/or a capacity of the at least one user. Further, the at least one score may include a metric score, a resilience score, a factor score (resilience factor score), an attribute score (resilience attribute score), etc. Further, the competency may include a resilience competency, a motivation competency, etc. Further, the at least one score may accurately quantify the capacity of the at least one user in the at least one psychological skill. Further, the at least one psychological skill may include an emotional resilience skill, a motivation skill, a cognitive skill, an emotion regulation skill, a cognitive reframing skill, a mindfulness skill, a self-compassion skill, an adaptability skill, a social support skill, a problem-solving skill, a seeking meaning skill, a motivation skill, etc. Further, at 210, the method 200 may include generating, using the processing device, at least one profile associated with the at least one psychological skill for the at least one user based on the generating of the at least one score for the at least one metric. Further, the at least one profile may provide granular insights into levels of the competency across attributes and include targeted training recommendations customized to needs of the at least one user. Further, the at least one profile may include a resilience profile. Further, at 212, the method 200 may include transmitting, using the communication device, the at least one profile to the at least one device. Further, at 214, the method 200 may include storing, using a storage device (such as a storage device 906), at least one assessment data may include the at least one response and the at least one score for the at least one metric, and the at least one profile.

Further, in an embodiment, the at least one user device may include at least one output device. Further, the at least one output device may be configured for presenting the at least one prompt to the at least one user based on the at least one prompt information, Further, the presenting of the at least one prompt may include presenting the at least one prompt information. Further, the at least one user device may include at least one sensor may include a motion sensor, an image sensor, a microphone, a eye tracking sensor, etc. Further, the at least one sensor may be configured for detecting at least one of a gesture, a movement, an utterance, a facial expression, a physiological response, a neurological response, and an emotional response. Further, the at least one device may be configured for generating the at least one response based on the detecting of at least one of the gesture, the movement, the utterance, the facial expression, the physiological response, the neurological response, and the emotional response. Further, the response may be a reaction. Further, the at least one sensor may be configured for generating the at least one response based on the detecting

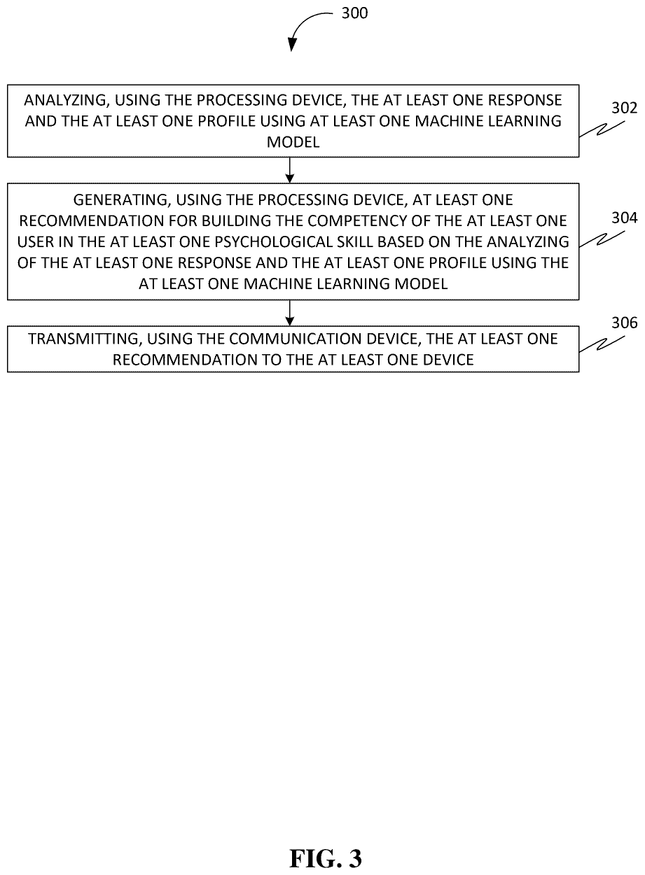

FIG. 3 is a flow chart of a method 300 for facilitating assessing psychological skills of users, in accordance with some embodiments. Accordingly, at 302, the method 300 may include analyzing, using the processing device, the at least one response and the at least one profile using at least one machine learning model. Further, the at least one machine learning model may include at least one gradient-boosting decision tree model. Further, the at least one gradient-boosting decision tree model may be trained on at least one training data for learning at least one of a relationship and an interaction between one or more assessment attributes and one or more optimal recommendations. Further, the one or more assessment attributes self-efficacy, coping skills, adaptability, perseverance, purpose, relationships, etc.

Further, at 304, the method 300 may include generating, using the processing device, at least one recommendation for building the competency of the at least one user in the at least one psychological skill based on the analyzing of the at least one response and the at least one profile using the at least one machine learning model. Further, the at least one recommendation may include at least one training recommendation. Further, the at least one recommendation may include personalized guidance for the at least one user based on at least one profile.

Further, at 306, the method 300 may include transmitting, using the communication device, the at least one recommendation to the at least one device.

Further, in some embodiments, the at least one machine learning model may be an ensemble of at least 100 decision trees. Further, a maximum decision tree depth for the at least one machine learning model may be at least 15.

Further, in some embodiments, the method 300 may include determining, using the processing device, at least one task associated with the at least one user based on the analyzing of the at least one profile and the at least one response using a natural language processing model. Further, the method 300 may include determining, using the processing device, at least one task information associated with the at least one task based on the determining of the at least one task. Further, the determining of the at least one task information may be initiated based on at least one predefined condition. Further, at least one contextual variable based on the at least one predefined condition further represents a condition relevant to the determining of the at least one task information. Further, the at least one contextual variable may include a physical state of the at least one device. Further, the at least one device comprises at least one sensor for generating the physical state of the at least one user device. Further, the method 300 may include analyzing, using the processing device, the at least one task information. Further, the method 300 may include modifying, using the processing device, the at least one score for the at least one metric based on the analyzing of the at least one task information. Further, the method 300 may include generating, using the processing device, at least one modified score for the at least one metric based on the modifying. Further, the generating of the at least one profile may be based on the generating of the at least one modified score for the at least one metric. Further, the at least one task may be one or more actions that need to be performed by the at least one user using the at least one device for building the competency of the at least one user.

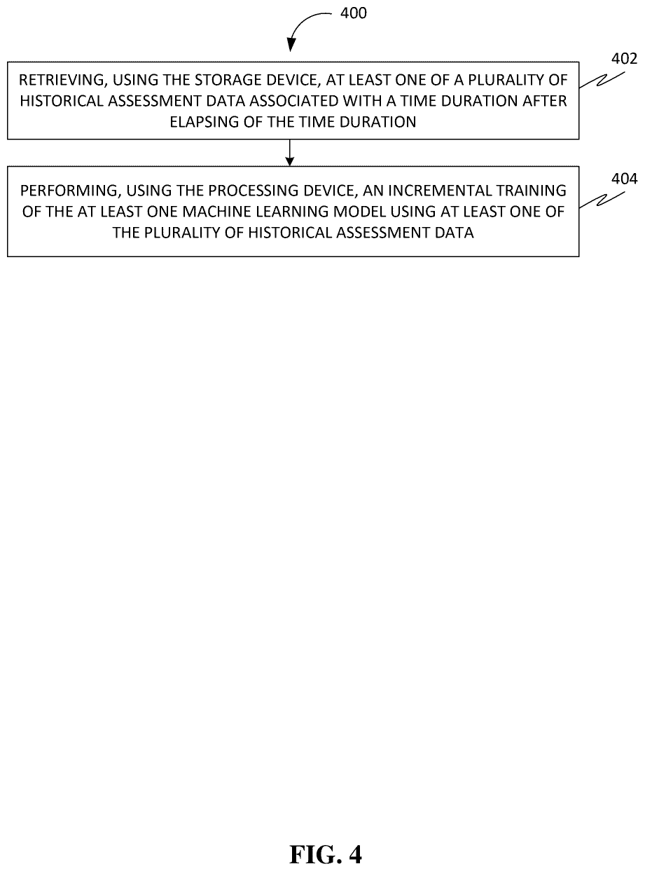

FIG. 4 is a flow chart of a method 400 for facilitating assessing psychological skills of users, in accordance with some embodiments. Accordingly, at 402, the method 400 may include retrieving, using the storage device, at least one of a plurality of historical assessment data associated with a time duration after elapsing of the time duration. Further, the time duration may be a day, a week, a month, a year, etc. Further, at 404, the method 400 may include performing, using the processing device, an incremental training of the at least one machine learning model using at least one of the plurality of historical assessment data. Further, the analyzing of the at least one response and the at least one profile using the at least one machine learning model may be based on the performing of the incremental training of the at least one machine learning model. Further, the time duration may include a week. Further, the incremental training may include a partial model fitting of the at least one machine learning model.

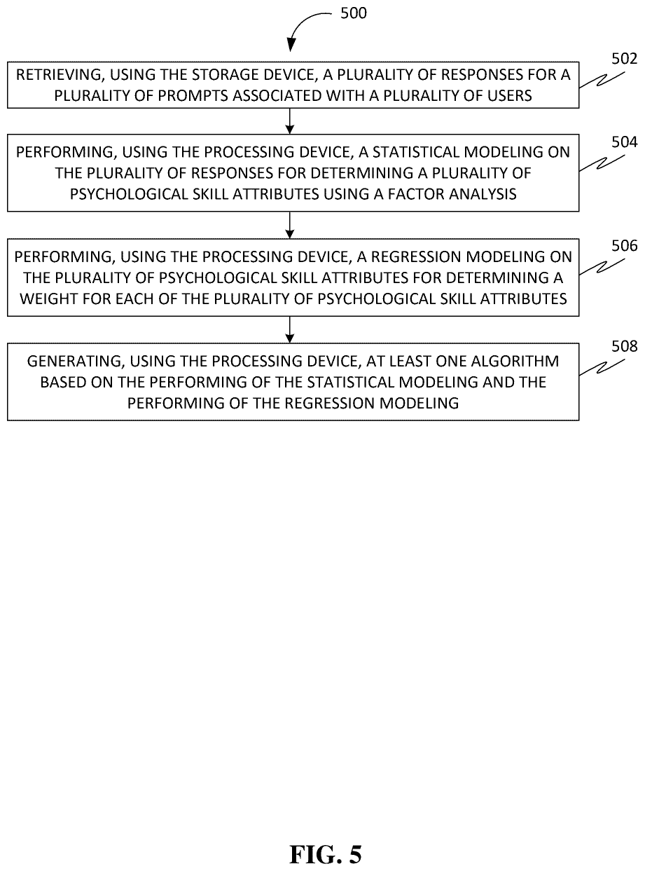

FIG. 5 is a flow chart of a method 500 for facilitating assessing psychological skills of users, in accordance with some embodiments. Accordingly, at 502, the method 500 may include retrieving, using the storage device, a plurality of responses for a plurality of prompts associated with a plurality of users. Further, at 504, the method 500 may include performing, using the processing device, a statistical modeling on the plurality of responses for determining a plurality of psychological skill attributes using a factor analysis. Further, the plurality of psychological skill attributes may include self-efficacy, coping skills, adaptability, perseverance, purpose, and relationships. Further, at 506, the method 500 may include performing, using the processing device, a regression modeling on the plurality of psychological skill attributes for determining a weight for each of the plurality of psychological skill attributes. Further, the weight of each of the plurality of psychological skill attributes may maximize predictive validity against external resilience criteria. Further, at 508, the method 500 may include generating, using the processing device, at least one algorithm based on the performing of the statistical modeling and the performing of the regression modeling. Further, the analyzing of the at least one response may include analyzing the at least one response using the at least one algorithm. Further, the generating of the at least one score may be based on the analyzing of the at least one response using the at least one algorithm.

Further, in some embodiments, the analyzing of the at least one response using the at least one algorithm may include evaluating the competency of the at least one user against each of the plurality of psychological skill attributes based on the at least one response. Further, the analyzing of the at least one response using the at least one algorithm may include scoring each of the plurality of psychological skill attributes based on the evaluating. Further, the analyzing of the at least one response using the at least one algorithm may include computing a weighted average score for the plurality of psychological skill attributes based on the weight of each of the plurality of psychological skill attributes and the scoring. Further, the generating of the at least one score for the at least one metric may be based on the computing. Further, the weighted average score may represent a final score of the psychological skills of the at least one user. Further, in an instance, the weighted average score may include a 0-100 ERMQ score.

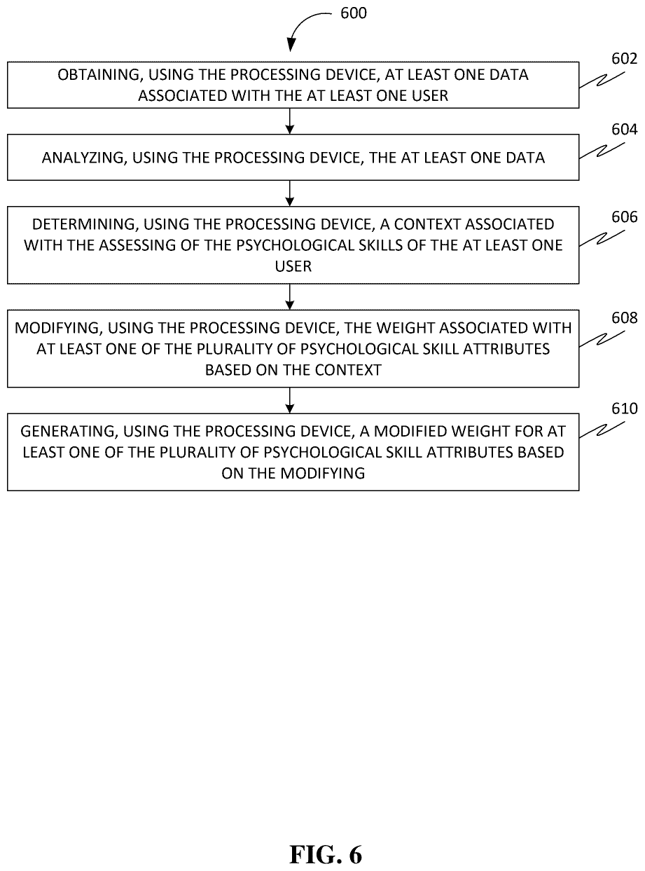

FIG. 6 is a flow chart of a method 600 for facilitating assessing psychological skills of users, in accordance with some embodiments. Accordingly, at 602, the method 600 may include obtaining, using the processing device, at least one data associated with the at least one user. Further, the at least one data may include a personal data, a professional data, a financial data, etc., of the at least one user. Further, at 604, the method 600 may include analyzing, using the processing device, the at least one data. Further, at 606, the method 600 may include determining, using the processing device, a context associated with the assessing of the psychological skills of the at least one user. Further, the context may include a subject and a use case associated with the at least one user. Further, at 608, the method 600 may include modifying, using the processing device, the weight associated with at least one of the plurality of psychological skill attributes based on the context. Further, at 610, the method 600 may include generating, using the processing device, a modified weight for at least one of the plurality of psychological skill attributes based on the modifying. Further, the computing of the weighted average score for the plurality of psychological skill attributes may be based on the modified weight of at least one of the plurality of psychological skill attributes.

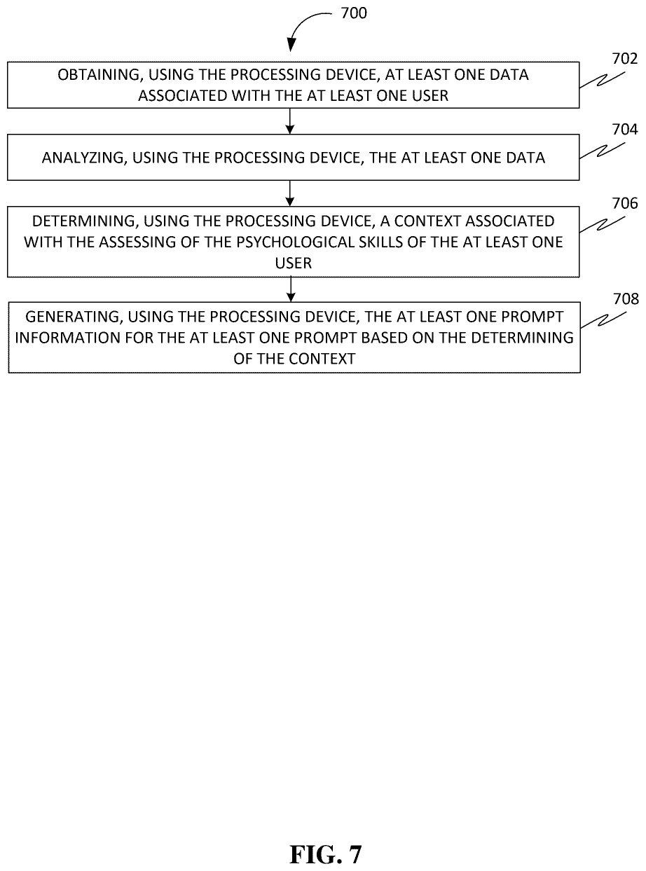

FIG. 7 is a flow chart of a method 700 for facilitating assessing psychological skills of users, in accordance with some embodiments. Accordingly, at 702, the method 700 may include obtaining, using the processing device, at least one data associated with the at least one user. Further, the at least one data may include a personal data, a professional data, a financial data, etc., of the at least one user. Further, at 704, the method 700 may include analyzing, using the processing device, the at least one data. Further, at 706, the method 700 may include determining, using the processing device, a context associated with the assessing of the psychological skills of the at least one user. Further, at 708, the method 700 may include generating, using the processing device, the at least one prompt information for the at least one prompt based on the determining of the context.

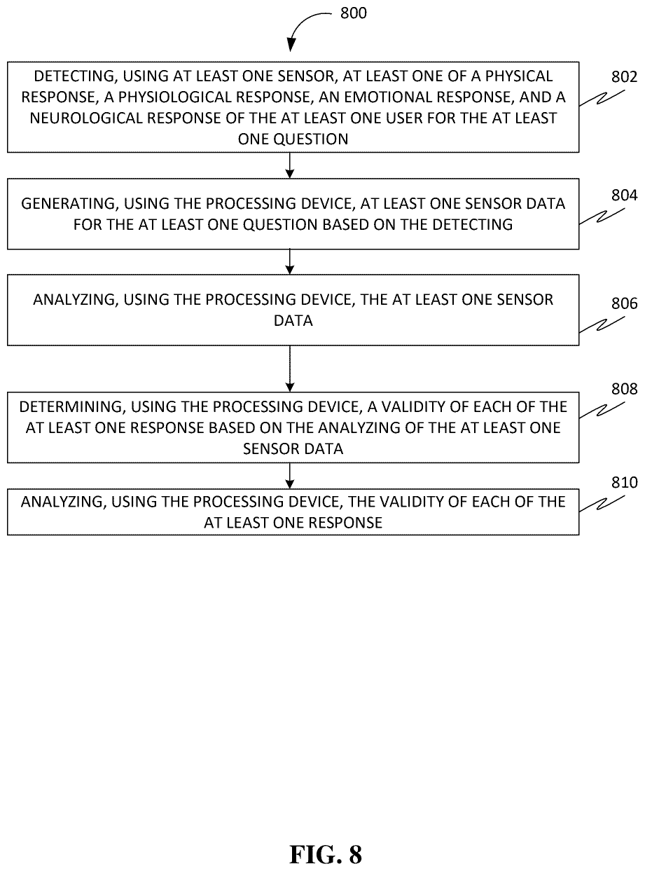

FIG. 8 is a flow chart of a method 800 for facilitating assessing psychological skills of users, in accordance with some embodiments. Accordingly, the at least one prompt information may include at least one questionnaire. Further, the at least one questionnaire may include at least one question and a plurality of answer options for each of the at least one question. Further, the at least one response may include a selection of answer option from the plurality of answer options for at least one of the at least one question. Further, at 802, the method 800 may include detecting, using at least one sensor (such as at least one sensor 1002), at least one of a physical response, a physiological response, an emotional response, and a neurological response of the at least one user for the at least one question. Further, the physical response may include increased heart rate, tense muscles, changes in breathing patterns, etc. Further, the physiological response may include pupil dilation, sweating, digestive changes, etc. Further, the emotional response may include excitement, anxiety, confidence, doubt, etc. Further, the neurological response may include increased activation in decision-making areas, changes in brain waves, processing speed changes, etc. Further, the at least one sensor may include a heart rate monitor, an Electromyography (EMG) sensor, a respiration rate monitor, a pupilometer (for measuring pupil dilation), a galvanic skin response (GSR) sensor, a digestive activity sensor, a facial expression analysis software, a voice stress analysis software, an Electroencephalography (EEG) headset, a Functional Magnetic Resonance Imaging (FMRI) sensor, a reaction time measurement device, etc. Further, at 804, the method 800 may include generating, using the processing device, at least one sensor data for the at least one question based on the detecting. Further, at 806, the method 800 may include analyzing, using the processing device, the at least one sensor data. Further, at 808, the method 800 may include determining, using the processing device, a validity of each of the at least one response based on the analyzing of the at least one sensor data. Further, the validity may indicate whether the at least one response is genuine or not. Further, the validity may include a positive validity and a negative validity. Further, at 810, the method 800 may include analyzing, using the processing device, the validity of each of the at least one response. Further, the generating of the at least one score for the at least one metric associated with the at least one psychological skill may be based on the analyzing of the validity of each of the at least one response.

Further, in some embodiments, the analyzing of the at least one response further may include analyzing the at least one response using at least one behavioral model and at least one natural language processing (NLP) model. Further, the at least one behavioral model and the at least one NLP model are separately trained on a plurality of training responses. Further, the plurality of training responses may include responses of users to questions. Further, the at least one behavioral model may include Big Five Personality Model, Cognitive Behavioral Therapy (CBT) Models, Transactional Analysis (TA), Emotional Intelligence (EI) model, etc. Further, the at least one behavioral model may include a machine learning behavioral model. Further, the at least one behavioral model may include a psychological behavioral model, a physical behavioral model, a neurological behavioral model, an emotional behavioral model, etc. Further, the behavioral model comprises at least one machine learning model for the at least one user that identifies an anomaly in a behavior (a physical behavior, a psychological behavior, a neurological behavior, an emotional behavior, etc.) based on past behavioral patterns of the at least one user. Further, the at least one behavioral model may be trained using one or more behavioral characteristics of the at least one user. Further, the one or more behavioral characteristics may include a breathing rate, a heart rate, a pupil dilation, a sweating, a gesture, a movement, an expression, a facial expression, etc. Further, the at least one machine learning model may include a Bayesian hierarchical regression model for identifying the anomaly in the behavior.

Further, the analyzing of the at least one response using the at least one behavioral model and the at least one NLP model may include obtaining at least one first output from the at least one behavioral model by inputting the at least one response to the at least one behavioral model. Further, the analyzing of the at least one response using the at least one behavioral model and the at least one NLP model may include obtaining at least one second output from the at least one NLP model by inputting the at least one response to the at least one NLP model. Further, the analyzing of the at least one response using the at least one behavioral model and the at least one NLP model may include combining the at least one first output and the at least one second output. Further, the generating of the at least one score for the at least one metric may be based on the combining.

Further, in an embodiment, the method 800 may include initiating, using the processing device, at least one session for the at least one user. Further, the detecting of at least one of the physical response, the physiological response, the emotional response, and the neurological response of the at least one user for the at least one question may be further based on the initiating. Further, the at least one session may include an assessment session.

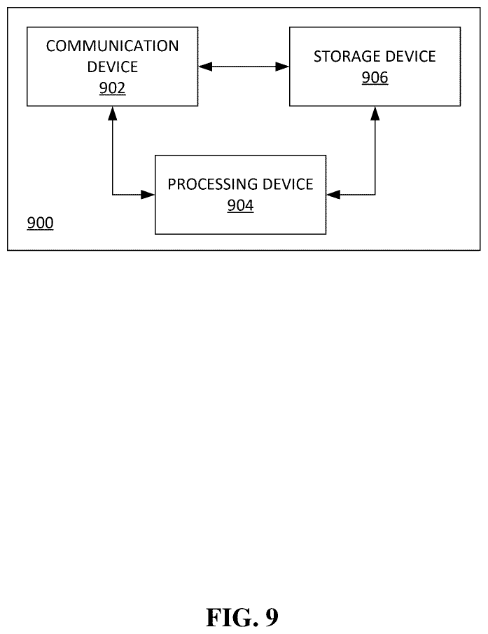

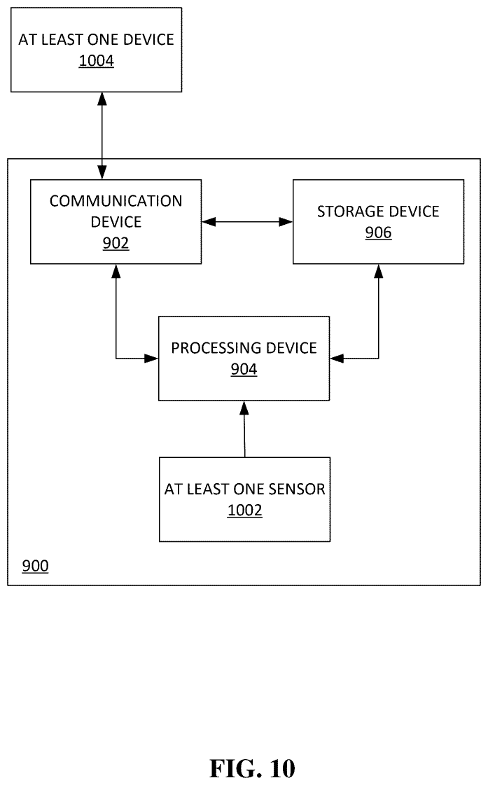

FIG. 9 is a block diagram of a system 900 for facilitating assessing psychological skills of users, in accordance with some embodiments. Accordingly, the system 900 may include a communication device 902 configured for transmitting at least one prompt information of at least one prompt to at least one device 1004 (as shown in FIG. 10). Further, the communication device 902 may be configured for receiving at least one response of at least one user for the at least one prompt from the at least one device 1004. Further, the communication device 902 may be configured for transmitting at least one profile to the at least one device 1004.

Further, the system 900 may include a processing device 904 communicatively coupled with the communication device 902. Further, the processing device 904 may be configured for analyzing the at least one response. Further, the processing device 904 may be configured for generating at least one score for at least one metric associated with at least one psychological skill based on the analyzing of the at least one response. Further, the at least one score for the at least one metric quantifies a competency of the at least one user in the at least one psychological skill. Further, the processing device 904 may be configured for generating the at least one profile associated with the at least one psychological skill for the at least one user based on the generating of the at least one score for the at least one metric.

Further, the system 900 may include a storage device 906 communicatively coupled with the processing device 904. Further, the storage device 906 may be configured for storing at least one assessment data may include the at least one response and the at least one score for the at least one metric, and the at least one profile.

Further, in some embodiments, the processing device 904 may be configured for analyzing the at least one response and the at least one profile using at least one machine learning model. Further, the at least one machine learning model may include at least one gradient-boosting decision tree model. Further, the at least one gradient-boosting decision tree model may be trained on at least one training data for learning at least one of a relationship and an interaction between one or more assessment attributes and one or more optimal recommendations. Further, the processing device 904 may be configured for generating at least one recommendation for building the competency of the at least one user in the at least one psychological skill based on the analyzing of the at least one response and the at least one profile using the at least one machine learning model. Further, the storage device 906 may be configured for transmitting the at least one recommendation to the at least one device 1004.

Further, in some embodiments, the at least one machine learning model may be an ensemble of at least 100 decision trees. Further, a maximum decision tree depth for the at least one machine learning model may be at least 15.

Further, in some embodiments, the storage device 906 may be configured for retrieving at least one of a plurality of historical assessment data associated with a time duration after elapsing of the time duration. Further, the processing device 904 may be configured for performing an incremental training of the at least one machine learning model using at least one of the plurality of historical assessment data. Further, the analyzing of the at least one response and the at least one profile using the at least one machine learning model may be based on the performing of the incremental training of the at least one machine learning model.

Further, in some embodiments, the storage device 906 may be configured for retrieving a plurality of responses for a plurality of prompts associated with a plurality of users. Further, the processing device 904 may be configured for performing a statistical modeling on the plurality of responses for determining a plurality of psychological skill attributes using a factor analysis. Further, the processing device 904 may be configured for performing a regression modeling on the plurality of psychological skill attributes for determining a weight for each of the plurality of psychological skill attributes. Further, the processing device 904 may be configured for generating at least one algorithm based on the performing of the statistical modeling and the performing of the regression modeling. Further, the analyzing of the at least one response may include analyzing the at least one response using the at least one algorithm. Further, the generating of the at least one score may be based on the analyzing of the at least one response using the at least one algorithm.

Further, in some embodiments, the analyzing of the at least one response using the at least one algorithm may include evaluating the competency of the at least one user against each of the plurality of psychological skill attributes based on the at least one response. Further, the analyzing of the at least one response using the at least one algorithm may include scoring each of the plurality of psychological skill attributes based on the evaluating. Further, the analyzing of the at least one response using the at least one algorithm may include computing a weighted average score for the plurality of psychological skill attributes based on the weight of each of the plurality of psychological skill attributes and the scoring. Further, the generating of the at least one score for the at least one metric may be based on the computing.

Further, in some embodiments, the processing device 904 may be configured for obtaining at least one data associated with the at least one user. Further, the processing device 904 may be configured for analyzing the at least one data. Further, the processing device 904 may be configured for determining a context associated with the assessing of the psychological skills of the at least one user. Further, the processing device 904 may be configured for modifying the weight associated with at least one of the plurality of psychological skill attributes based on the context. Further, the processing device 904 may be configured for generating a modified weight for at least one of the plurality of psychological skill attributes based on the modifying. Further, the computing of the weighted average score for the plurality of psychological skill attributes may be based on the modified weight of at least one of the plurality of psychological skill attributes.

Further, in some embodiments, the processing device 904 may be configured for obtaining at least one data associated with the at least one user. Further, the processing device 904 may be configured for analyzing the at least one data. Further, the processing device 904 may be configured for determining a context associated with the assessing of the psychological skills of the at least one user. Further, the processing device 904 may be configured for generating the at least one prompt information for the at least one prompt based on the determining of the context.

Further, in some embodiments, the at least one prompt information may include at least one questionnaire. Further, the at least one questionnaire may include at least one question and a plurality of answer options for each of the at least one question. Further, the at least one response may include a selection of answer option from the plurality of answers option for at least one of the at least one question. Further, the system 900 may include at least one sensor 1002 (as shown in FIG. 10) communicatively coupled with the processing device 904. Further, the at least one sensor 1002 may be configured for detecting at least one of a physical response, a physiological response, an emotional response, and a neurological response of the at least one user for the at least one question. Further, the processing device 904 may be configured for generating at least one sensor data for the at least one question based on the detecting. Further, the processing device 904 may be configured for analyzing the at least one sensor data. Further, the processing device 904 may be configured for determining a validity of each of the at least one response based on the analyzing of the at least one sensor data. Further, the processing device 904 may be configured for analyzing the validity of each of the at least one response. Further, the generating of the at least one score for the at least one metric associated with the at least one psychological skill may be based on the analyzing of the validity of each of the at least one response.

Further, in some embodiments, the analyzing of the at least one response may include analyzing the at least one response using at least one behavioral model and at least one natural language processing (NLP) model. Further, the at least one behavioral model and the at least one NLP model are separately trained on a plurality of training responses. Further, the analyzing of the at least one response using the at least one behavioral model and the at least one NLP model may include obtaining at least one first output from the at least one behavioral model by inputting the at least one response to the at least one behavioral model. Further, the processing device 904 may be configured for obtaining at least one second output from the at least one NLP model by inputting the at least one response to the at least one NLP model. Further, the processing device 904 may be configured for combining the at least one first output and the at least one second output. Further, the generating of the at least one score for the at least one metric may be based on the combining.

FIG. 10 is a block diagram of the system 900 for facilitating assessing psychological skills of users, in accordance with some embodiments.

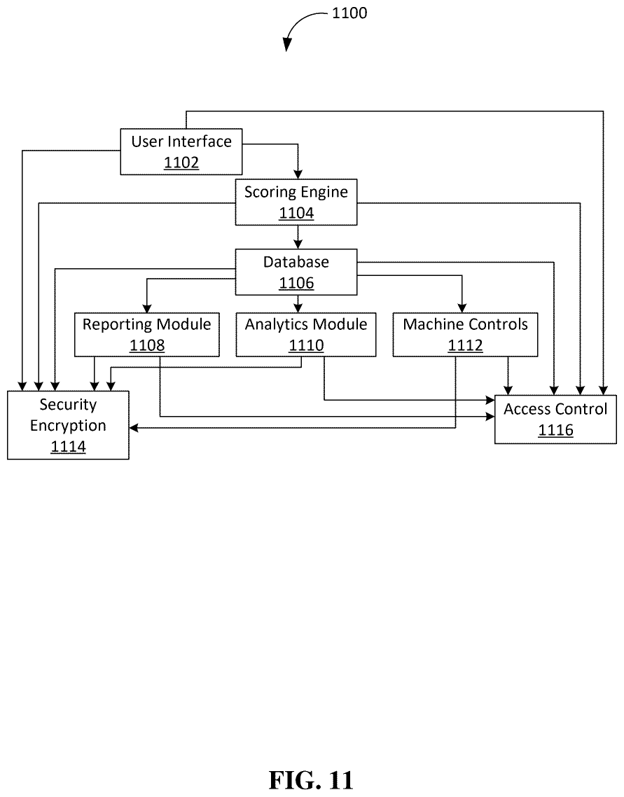

FIG. 11 illustrates a system 1100 for facilitating assessing psychological skills of users, in accordance with some embodiments. Accordingly, the system 1100 may be an emotional resilience assessment system. Within FIG. 11, the system may include primary components and their interconnections, showcasing how each module interacts and contributes to the system's functionality. From initial user interactions to intricate processes behind the scenes, ‘System Architecture Workflow’ associated with the system offers a comprehensive insight into the system's mechanics and its end-to-end journey. FIG. 11 shows a detailed visualization of the overarching system design and its operational sequence.

Further, the system 1100 may be configured to display a user interface 1102. Further, the system 1100 may include a scoring engine 1104 and a database 1106 communicatively coupled to the scoring engine 1104. Further, the system 1100 may include a reporting module 1108 and an analytics module 1110. Further, the database 1106 may be associated with machine controls 1112. Further, the database 1106 and the scoring engine 1104 may be associated with security encryption 1114. Further, at 1116, the system 1100 may be configured to perform access control.

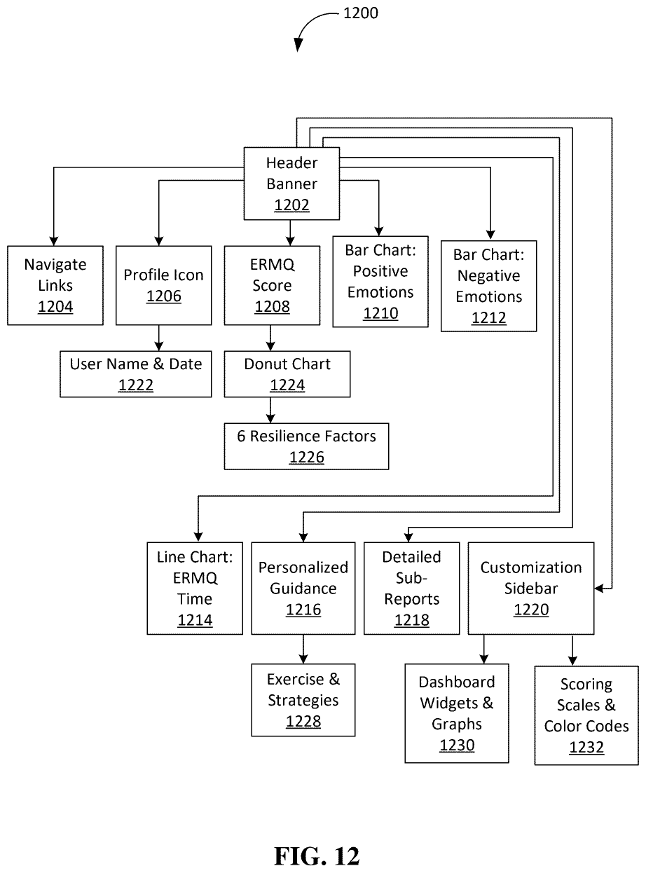

FIG. 12 illustrates a personalized resilience assessment report 1200, in accordance with some embodiments. Accordingly, the personalized resilience assessment report 1200 may be seen by a user after completing the questionnaire. FIG. 12 illustrates insights, visualizations, and customization options associated with the personalized resilience assessment report. Further, the personalized resilience assessment report 1200 may include a header banner 1202. Further, the personalized resilience assessment report 1200 may include navigation links 1204 in the header that allows users to access other views like the questionnaire, account settings, knowledge base articles, etc. In a top right corner of a user interface displaying the personalized resilience assessment report 1200, the personalized resilience assessment report 1200 may include a profile icon 1206 indicating the name of the user who took the assessment and the date, at 1222, when it was completed. This provides context. Further, at 1208, the personalized resilience assessment report 1200 may display a composite ERMQ or Emotional Resilience & Motivation Quotient score which summarizes the user's resilience capacity based on their responses. The ERMQ score may be shown with descriptive text interpreting what the number means. Further, below the ERMQ score in the user interface, the personalized resilience assessment report may include a donut chart 1224 illustrating the breakdown of the ERMQ across the 6 constituent resilience factors 1226—self-efficacy, relationships, purpose, adaptability, coping skills, and motivation. Each factor can have a color coding. The donut chart 1224 indicates the contribution of each factor. Further, the personalized resilience assessment report 1200 may include two bar charts 1212 and 1214 comparing the user's emotional scaling scores across the positive and negative emotion spectra. The scores for emotions like joy, passion, frustration, anxiety, etc. can be plotted for side-by-side comparison. Further, at 1214, the personalized resilience assessment report 1200 may include a line chart that plots the user's ERMQ scores over time across the different occasions they have taken the assessment. This displays trends and progress. Further, at 1216, in a personalized guidance section, the personalized resilience assessment report 1200 may show text or icons linking to exercises and strategies 1228 and training recommendations that are tailored for the user based on their assessment responses and resilience profile. These helps build their competencies. Below, the user interface may display icons for different sections that can be clicked to access more detailed sub-reports 1218 on each part of the assessment like motivations, relationships, self-efficacy, etc. Further, at 1220, in a right customization sidebar of the user interface, the personalized resilience assessment report 1200 may illustrate options to customize and configure the dashboard-selecting/rearranging dashboard widgets and graphs 1230, changing their design, and customizing scoring scales and color codes 1232. based on user preferences. This allows personalization. The dashboard visualizations, insights, and customizations may provide the users with an engaging, interactive assessment report that makes the experience personalized.

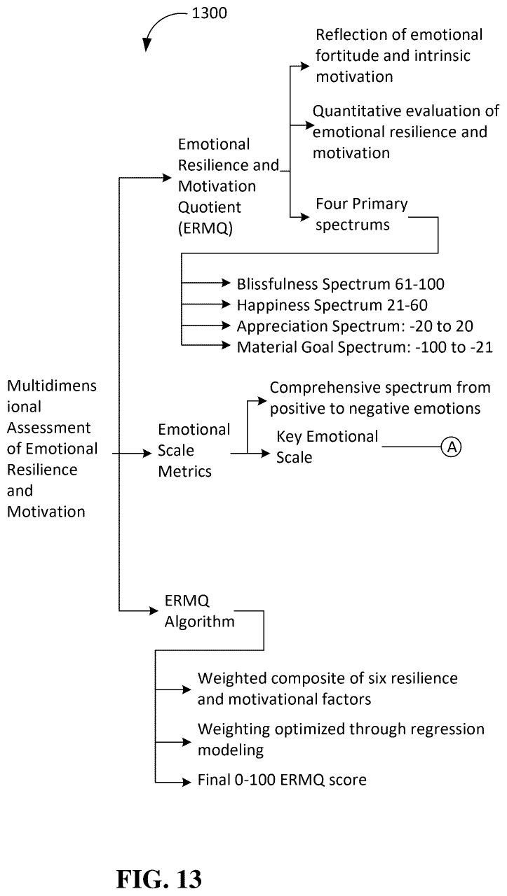

FIG. 13 illustrates a dashboard view of an exemplary personalized resilience assessment report 1300, in accordance with some embodiments. Accordingly, the personalized resilience assessment report 1300 may be seen by users after completing a questionnaire. Upon completing the ERMQ, the users are greeted with a user-friendly dashboard that prominently displays their ER score, offering a snapshot of their emotional resilience. This score is contextualized with graphical comparisons to average scores and a breakdown of resilience components. Users can identify their strengths and areas for improvement, track score progression over time, and receive personalized recommendations to bolster resilience. The dashboard also aligns findings with APA guidelines and provides insights into the legal implications of emotional well-being. Convenient features like secure sharing options and report exporting enhance the user experience, while feedback mechanisms ensure continuous improvement of the assessment tool.

Further, the disclosed system may be configured for performing multi dimensional assessment of emotional resilience and motivation of a user based on ERMQ, emotional scale metric, and an ERMQ algorithm. Further, the ERMQ may be a reflection of emotional fortitude and intrinsic motivation. Further, the ERMQ may facilitate quantitative evaluation of emotional resilience and motivation. Further, the ERMQ may be associated with four primary spectrums. Further, the four primary spectrums may include a blissfulness spectrum 61-100, a happiness spectrum 21-60, an appreciation spectrum: −20 to 20, and a material goal spectrum: −100 to −21. Further, the emotional scale metric may provide comprehensive spectrum from positive to negative emotions. Further, the emotional scale metric may be associated with key emotional scale. Further, the key emotional scale may include joy/appreciation/empowerment: 100, passion: 90, enthusiasm: 80, optimism: 70, hopefulness: 60, contentment: 50, doubt: 40, boredom: 30, disappointment: 20, pessimism: 10, worry: 0, overwhelming: −10, frustration: −20, discouragement: −30, insecurity/unworthiness: −40, fear/despair: −50, anger/revenge: −60, blame: −70, jealousy: −80, and hatred: −100. Further, the ERMQ algorithm may be based on weighted composite of six resilience and motivational factors. Further, the ERMQ algorithm may facilitate optimizing weighting through regression modeling and provide a final 0-100 ERMQ score.

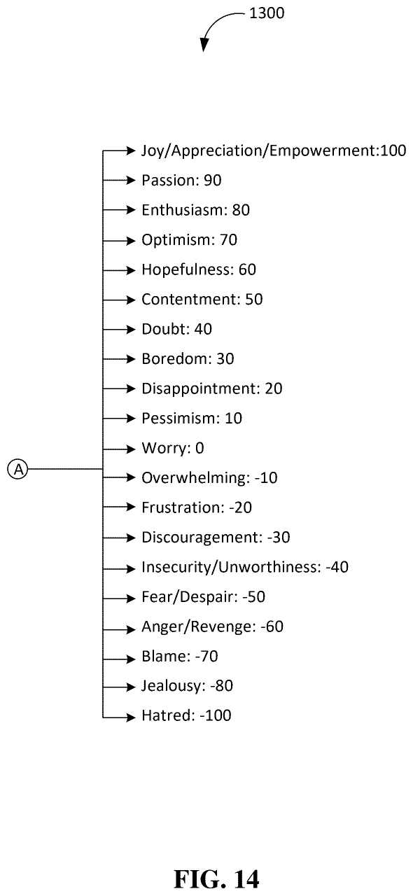

FIG. 14 is a continuation of the dashboard view of the exemplary personalized resilience assessment report 1300, in accordance with some embodiments.

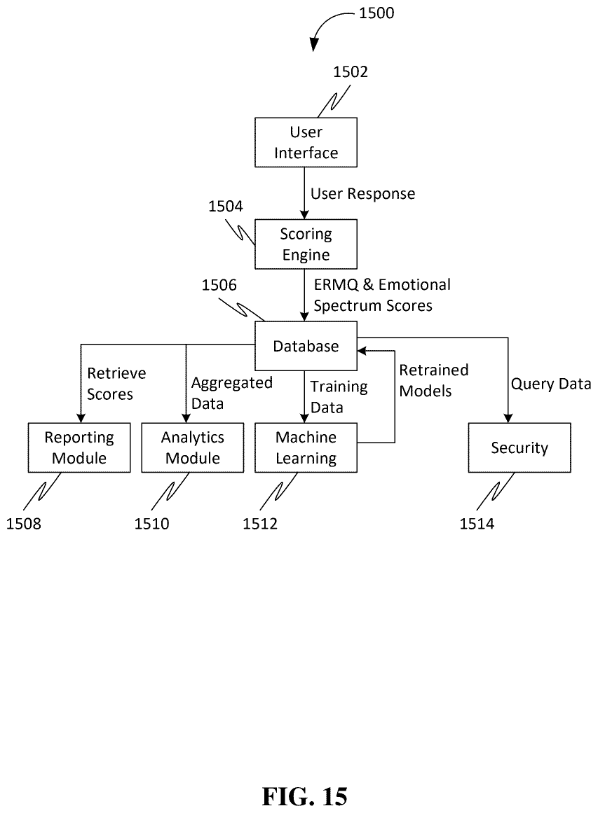

FIG. 15 is a schematic of a system 1500 for facilitating assessing psychological skills of users, in accordance with some embodiments. Accordingly, at 1502, the system 1500 may include a user Interface (UI) module that allows users to take an interactive resilience questionnaire. The UI is designed as a responsive website or mobile app with multimedia features for engagement. It captures the subject's responses. Further, at 1504, the system 1500 may include a scoring engine module that encapsulates the proprietary algorithms developed to analyze the subject's responses and compute their ERMQ and emotional spectrum scores. The scoring logic can be packaged as a software library or module exposing APIs. Further, at 1506, the system 1500 may include a database module that contains all assessment data including user responses and computed scores. Further, the database module allows efficient storage and retrieval of the data for reporting and analytics. The database may utilize SQL, NoSQL, cloud, etc. based on scalability and performance needs. Further, at 1508, the system 1500 may include a reporting module that generates detailed, personalized assessment reports and resilience profiles for each subject by querying their data from the database. Further, the reporting module implements interactive report dashboards. Further, at 1510, the system 1500 may include an analytics module that leverages techniques like statistical modeling, predictive analytics, and data mining on aggregated assessment data to gain insights that inform tool optimization and research. Further, at 1512, the system 1500 may include a machine learning module that trains ML algorithms on the database of responses to build models for the recommendation engine that generates personalized guidance for users based on their resilience profiles. The ML models can be iteratively retrained on new data. Further, at 1514, the system 1500 may include a security module that implements features like encryption, role-based access control, and authentication to protect sensitive user data. Encryption is applied for all data transfers.

Further, an architecture associated with the system 1500 may be hosted on scalable cloud infrastructure, leveraging autoscaling and load balancing to handle large user volumes. APIs enable interoperability between modules. Bidirectional arrows indicate data flows between components—from the UI through scoring, storage, reporting, and analytics. This illustrates how the different parts interact to deliver the end-to-end functionality.

FIG. 15 illustrates the key components and flow of the computer-implemented system for automated emotional resilience assessment, scoring, and reporting. The user interacts with the system through the user interface module, which presents the interactive questionnaire and captures the subject's responses. The responses are passed to the scoring engine, which calculates the proprietary ERMQ and emotional scaling scores using validated algorithms. These raw scores are stored in the database. The reporting module then retrieves the scores and generates a personalized resilience profile report for the subject. The system can be accessed remotely via web and mobile applications. Backend components include analytics, monitoring, and machine learning modules for system insights. Security components like encryption and access controls safeguard confidential data.

Further, the system 1500 may be configured for performing automated reporting. Further, the system 1500 may provide dashboards that allow customizing chart types, resilience factors displayed, and score ranges. Further, the system 1500 may allow administrators to configure user permissions, access controls, and manage usage analytics. Further, the system 1500 may be configured for performing cloud-based containerized deployment that enables scalable distribution.

FIG. 16 illustrates a table 1600 representing a comparative analysis between individuals who joined the Next League Program and those who did not, in accordance with some embodiments. Accordingly, the table 1600 may include 3 columns. Further, the 3 columns may include a criteria of assessment, group not joined, and group joined. Further, the column criteria of assessment may include 15 rows. Further, the 15 rows may include number of individuals, ERMQ score mean, ERMQ score SD, ERMQ score range, ER score mean, ER score SD, ER score range, test-retest reliability, Shapiro wilk p value, mann whitney U p-value, LMM intercept (SE) (p-value), LMM time coefficient (SE) (p-value), group variance, covariance, and time variance. Further, for the row number of individuals, column group not joined may include 798. Further, for the row number of individuals, column group joined may include 116. Further, for the row ERMQ score mean, column group not joined may include 27.59. Further, for the row ERMQ score mean, column group joined may include 57.20. Further, for the row ERMQ score SD, column group not joined may include 18.20. Further, for the row ERMQ score SD, column group joined may include 29.52. Further, for the row ERMQ score range, column group not joined may include −25 to 80. Further, for the row ERMQ score range, column group joined may include −5 to 100.

Further, for the row ER score mean, column group not joined may include 53.35. Further, for the row ER score mean, column group joined may include 65.36.

Further, for the row ER score SD, column group not joined may include 25.35. Further, for the row Er score SD, column group joined may include 26.28.

Further, for the row ER score range, column group not joined may include −50 to 100. Further, for the row ER score range, column group joined may include −14 to 100.

Further, for the row test retest reliability, column group not joined may not be applicable. Further, for the row test retest reliability, column group joined may include 0.80.

Further, for the row Shapiro-Wilk p-value, column group not joined may include ≈2.17×10\hat{ }−21. Further, for the row Shapiro-Wilk p-value, column group joined may include ≈1.05×10\hat{ }−11.

Further, for the row Mann-Whitney U p-value, column group not joined may not be applicable. Further, for the row Mann-Whitney U p-value, column group joined may include ≈9.28×10\hat{ }−57.

Further, for the row LMM Intercept (SE) (p-value), the column group not joined may not be applicable. Further, for the row LMM Intercept (SE) (p-value), the column group joined may include 46.451 (3.659) (<0.001).

Further, for the row LMM time coefficient (SE) (p-value), the column group not joined may not be applicable. Further, for the row LMM time coefficient (SE) (p-value), the column group joined may include 5.004 (1.449) (0.001).

Further, for the row group variance, the column group not joined may not be applicable. Further, for the row group variance, the column group joined may include 608.335.

Further, for the row co variance (initial score vs rate of change), the column group not joined may not be applicable. Further, for the row co variance (initial score vs rate of change), the column group joined may include −154.419.

Further, for the row time variance, the column group not joined may not be applicable. Further, for the row time variance, the column group joined may include 53.769.

FIG. 17 is a graphical representation of a grouped bar graph 1700 showing the mean ER and ERMQ scores for the “Not Joined” and “Joined” groups, in accordance with some embodiments.

FIG. 18 is a graphical representation of a box plot 1800 that provides a distributional perspective of the ER and ERMQ scores for both groups, in accordance with some embodiments.

FIG. 19 is a graphical representation of a line graph 1900 displaying high Emotional Resilience and Motivation Quotient (ERMQ) and Emotional Resilience (ER) scores of the first eight successful EB1A green card recipients from the EB1A Next League Program, in accordance with some embodiments.

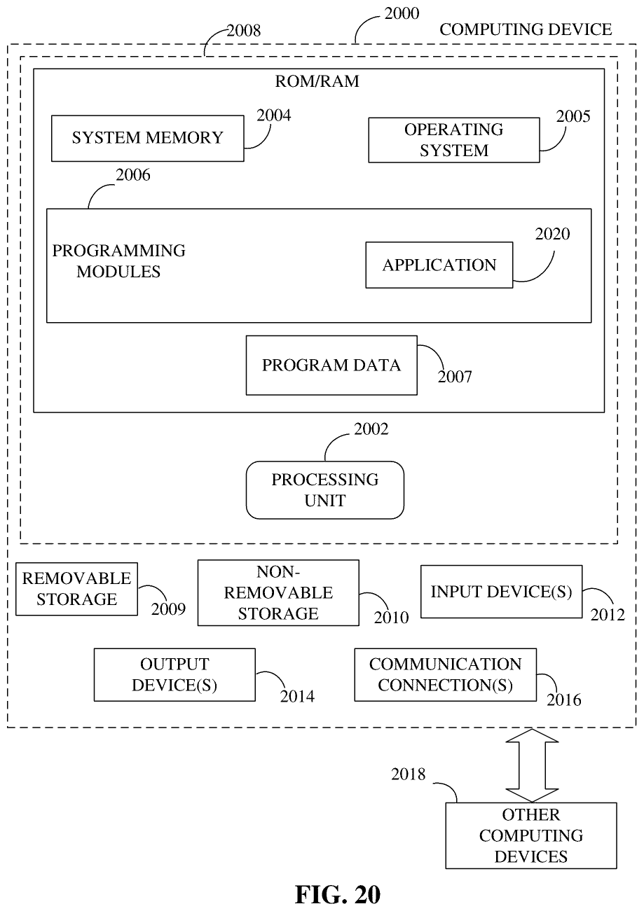

With reference to FIG. 20, a system consistent with an embodiment of the disclosure may include a computing device or cloud service, such as computing device 2000. In a basic configuration, computing device 2000 may include at least one processing unit 2002 and a system memory 2004. Depending on the configuration and type of computing device, system memory 2004 may comprise, but is not limited to, volatile (e.g. random-access memory (RAM)), non-volatile (e.g. read-only memory (ROM)), flash memory, or any combination. System memory 2004 may include operating system 2005, one or more programming modules 2006, and may include a program data 2007. Operating system 2005, for example, may be suitable for controlling computing device 2000's operation. In one embodiment, programming modules 2006 may include image-processing modules, machine learning modules, etc. Furthermore, embodiments of the disclosure may be practiced in conjunction with a graphics library, other operating systems, or any other application program and is not limited to any particular application or system. This basic configuration is illustrated in FIG. 20 by those components within a dashed line 2008.

Computing device 2000 may have additional features or functionality. For example, computing device 2000 may also include additional data storage devices (removable and/or non-removable) such as, for example, magnetic disks, optical disks, or tape. Such additional storage is illustrated in FIG. 20 by a removable storage 2009 and a non-removable storage 2010. Computer storage media may include volatile and non-volatile, removable, and non-removable media implemented in any method or technology for storage of information, such as computer-readable instructions, data structures, program modules, or other data. System memory 2004, removable storage 2009, and non-removable storage 2010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD), other optical storage, magnetic cassettes, magnetic tape, magnetic disk storage or other magnetic storage devices, or any other medium which can be used to store information and which can be accessed by computing device 2000. Any such computer storage media may be part of device 2000. Computing device 2000 may also have input device(s) 2012 such as a keyboard, a mouse, a pen, a sound input device, a touch input device, a location sensor, a camera, a biometric sensor, etc. Output device(s) 2014 such as a display, speakers, a printer, etc. may also be included. The aforementioned devices are examples and others may be used.

Computing device 2000 may also contain a communication connection 2016 that may allow device 2000 to communicate with other computing devices 2018, such as over a network in a distributed computing environment, for example, an intranet or the Internet. Communication connection 2016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2004, including operating system 2005. While executing on processing unit 2002, programming modules 2006 (e.g., application 2020 such as a media player) may perform processes including, for example, one or more stages of methods, algorithms, systems, applications, servers, databases as described above. The aforementioned process is an example, and processing unit 2002 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

### APPENDIX

### Key to Emotional Resilience and Motivation Quotient (ERMQ):

1. Why do you want to achieve the EB1A Green Card?
   a. To improve my financial condition and lifestyle. (−10)
   b. To prove my capabilities and gain recognition. (−5)
   c. To explore opportunities for growth and development. (5)
   d. To make a positive impact on the lives of others and contribute to society. (10)
2. How often do you express gratitude towards others?
   a. I focus more on my own endeavors. (−10)
   b. When someone does something nice for me. (−5)
   c. I recognize the importance of appreciation. (5)
   d. Expressing gratitude is integral to my values and interactions. (10)
3. What is your approach to philanthropy or giving back to the community?
   a. I will do it later as my goals take precedence. (−10)
   b. I sometimes contribute when it aligns with my resources and schedule. (−5)
   c. I proactively engage in charitable activities to support causes I care about. (5)
   d. I actively integrate philanthropy into my daily life, making a meaningful impact. (10)
4. How do you feel after achieving a significant goal?
   a. I feel satisfied but quickly shift focus to the next goal. (−5)
   b. I experience moments of happiness and acknowledge the achievement. (5)
   c. I appreciate the journey and express gratitude for the opportunities and support. (10)
   d. I feel a profound sense of bliss, connection, and gratitude for the accomplishment. (10)
5. How do you react to setbacks in achieving your goals?
   a. I become frustrated and doubt my abilities. (−10)
   b. I feel disappointed, but I try to maintain motivation and keep going. (−5)
   c. I view setbacks as learning opportunities and remain optimistic. (5)
   d. I embrace setbacks as chances for growth and to inspire others through my experiences. (10)
6. How do you approach your work or professional endeavors?
   a. I primarily focus on achieving professional goals and success. (−5)
   b. I appreciate the opportunities my work provides and express gratitude. (5)
   c. I strive to make a positive impact and contribute to the welfare of others. (5)
   d. My work is driven by a deep sense of purpose and the desire to give back. (10)
7. How do you view your professional achievements?
   a. As stepping stones towards my individual goals. (−5)
   b. As sources of personal happiness and fulfillment. (5)
   c. As opportunities to express gratitude towards my support system. (10)
   d. As avenues to experience joy, and fulfillment, and positively impact others. (10)
8. How do you handle criticism or feedback?
   a. I get demotivated. (−10)
   b. I try to learn from it, but it affects my mood. (−5)
   c. I appreciate it as an opportunity to improve and express gratitude for the feedback. (5)
   d. I view it as a chance to grow and help others facing similar challenges. (10)
9. What motivates you to keep pursuing your goals?
   a. The success and status associated with achievement. (−10)
   b. The happiness and satisfaction derived from accomplishing my goals. (5)
   c. The opportunity to express gratitude and appreciate the journey. (10)
   d. The desire to create a positive impact and contribute to the well-being of others. (10)
10. What would you do if you achieve the EB1A Green Card?
   a. I would celebrate my success and enjoy the new opportunities it brings. (−5)
   b. I would feel happy and grateful for reaching this significant milestone. (5)
   c. I would appreciate the journey and express gratitude to everyone who supported me. (10)
   d. I would utilize my new position to create opportunities for others and make a positive impact. (10)

### Key to Emotional Resilience and Motivation Quotient (ERMQ) Spectrum:

1. Blissfulness Spectrum: Total score range of 61 to 100
2. Happiness Spectrum: Total score range of 21 to 60
3. Appreciation Spectrum: Total score range of −20 to 20
4. Material Goal Spectrum: Total score range of −100 to −21

### Key to Questionnaire on Emotional Resilience

1. What thoughts cross your mind when you think about your progress towards the EB1A Green Card?
   a. I see the progress as positive and confidence-building. (10)
   b. I worry, questioning if I'm doing enough. (3)
   c. I feel overwhelmed, thinking it's too much to handle. (−3)
   d. I fear that my efforts might not be enough and despair. (−10)
2. When you envision reaching your goal, what emotional response does that elicit?
   a. It fills me with excitement and enthusiasm. (10)
   b. It brings cautious hope. (3)
   c. It makes me question my abilities and fosters doubt. (−3)
   d. It sparks anger about the process's complexity. (−10)
3. What emotions arise when you reflect on your professional life?
   a. I feel a sense of empowerment and passion. (10)
   b. I find contentment in my situation. (3)
   c. I feel disinterested and uninspired. (−3)
   d. I battle feelings of insecurity and unworthiness. (−10)
4. What kind of thought process do you engage in when you encounter setbacks in your Green Card application?
   a. I perceive them as learning opportunities. (10)
   b. They instigate worry. (3)
   c. They lead me to blame myself or others. (−3)
   d. They provoke feelings of intense anger and revengeful thoughts. (−10)
5. How do you emotionally respond when you compare your progress with others?
   a. I remain optimistic about my journey. (10)
   b. I'm content with my own pace. (3)
   c. I feel disheartened and envious. (−3)
   d. I harbor intense feelings of hatred. (−10)
6. What emotional strategies do you employ to deal with the stress of the application process?
   a. I channel my stress into motivation for my goals. (10)
   b. I often feel stressed and overwhelmed. (−3)
   c. I battle fear and feelings of despair. (−7)
   d. I struggle with intense frustration. (−10)
7. How would you describe your emotional state when contemplating the likelihood of achieving your Green Card goal?
   a. I feel joy and strong appreciation. (10)
   b. I am hopeful and positive. (3)
   c. I am uncertain and doubtful. (−3)
   d. I lean towards pessimism and negativity. (−10)
8. How does the Green Card process impact your feelings towards your personal life?
   a. It stimulates my passion for personal growth. (10)
   b. It brings contentment and balance. (3)
   c. It leaves me feeling uninspired and bored. (−3)
   d. It makes me feel insecure and unworthy. (−10)
9. How does the thought of possible failure in your Green Card application affect you?
   a. It fuels my optimism and willingness to try again. (10)
   b. It breeds worry and fear. (3)
   c. It instills a feeling of defeat and discouragement. (−3)
   d. It evokes despair and fear. (−10)
10. What emotions do you experience when others succeed in their Green Card applications?
   a. I feel motivated and enthusiastic, considering their success as an inspiration. (10)
   b. I feel hopeful, believing that my time will come too. (3)
   c. I feel overwhelmed and start doubting my capabilities. (−3)
   d. I struggle with intense feelings of jealousy and hatred. (−10)

### Key to Emotional Scale:

1. Joy/Appreciation/Empowerment: 100
2. Passion: 90
3. Enthusiasm: 80
4. Optimism: 70
5. Hopefulness: 60
6. Contentment: 50
7. Doubt: 40
8. Boredom: 30
9. Disappointment: 20
10. Pessimism: 10
11. Worry: 0
12. Overwhelming: −10
13. Frustration: −20
14. Discouragement: −30
15. Insecurity/Unworthiness: −40
16. Fear/Despair: −50
17. Anger/Revenge: −60
18. Blame: −70
19. Jealousy: −80
20. Hatred: −100

### ASPECTS

1. A computer-implemented method for assessing emotional resilience and motivation through proprietary questionnaires and metrics comprising:
   Administering a set of stimulus items probing resilience and motivation attributes
   Processing responses through algorithms to generate an Emotional Resilience and Motivation Quotient (ERMQ) score
   Producing supplemental emotional scaling data
   Automated scoring and generation of personalized resilience profiles
   Customizable delivery, reporting, and guidance
   A multi-factor model and composite metric for quantifying emotional resilience and motivation comprising:
   Factors assessing self-efficacy, adaptability, perseverance, purpose, relationships, and motivational drives
   Weighting of factors optimized through statistical modeling of assessment data
   Formulations for calculating an overall ERMQ score summarizing resilience capacity
   Proprietary classification systems and scoring rubrics for responses
2. A computer system for implementing the emotional resilience assessment, scoring, and reporting.
3. The system of aspect 3, wherein the user interface comprises features for interactive delivery of questionnaire items, multimedia engagement, and intuitive navigation.
4. The system of aspect 3, wherein the architecture comprises modules for data storage, backup, disaster recovery, analytics, scoring, reporting, and machine learning algorithms.
5. The system of aspect 3, wherein security protocols include encryption, access controls, role-based permissions, and authentication.
6. A computer-implemented method for training machine learning algorithms to generate personalized resilience and motivation recommendations based on emotional resilience assessment data.
7. A system for collecting emotional resilience assessment data from a plurality of subjects, aggregating the data, and training machine learning models to produce personalized recommendations tailored to a subject's profile.
8. The system of aspect 3, wherein the graphical user interface comprises multimedia features for interactive delivery of assessment questions and motivational content.
9. The method of aspect 1, further comprises steps for encrypting assessment data, controlling user access to the system, and authenticating user identities.
10. The method of aspect 1, further comprising steps of:
   Presenting the set of interactive resilience assessment stimulus items to the subject via a user interface
   Receiving responses to the stimulus items through the user interface
11. The system of aspect 3, wherein the reporting module comprises features for selecting report sections, customizing scoring scales, and tailoring guidance recommendation models.
12. The system of aspect 3, further comprising application programming interfaces for integration with external systems.
13. The system of aspect 3, further comprising an administrator portal with configurable settings controlling access permissions, report parameters, and scoring models.
14. Preprocessing input data including encoding resilience scores and dimensionality reduction.
15. Training an ensemble model of decision trees using grid search hyperparameter optimization.
16. Iteratively retraining the machine learning model on new data

### REFERENCES

### Psychological Frameworks and Guidelines:

[1] American Psychological Association's guidelines on resilience and strength in adults, 2012.
[2] Mayer, J. D., & Salovey, P. (1997). What is emotional intelligence? In P. Salovey & D. J. Sluyter (Eds.), Emotional development and emotional intelligence: Educational implications (pp. 3-31). New York: Basic Books.

### Legislation and Treaties:

[3] Diamond v. Chakrabarty, 447 U.S. 303 (1980): A landmark U.S. Supreme Court case that established the patentability of genetically modified organisms.
[4] 1952 Patent Act: Overhauled the U.S. patent system, introducing concepts like non-obviousness and defining invention.
[5] Leahy-Smith America Invents Act: A U.S. federal statute from 2011, representing a significant change to the U.S. patent system.
[6] Patent Cooperation Treaty (PCT) (1970): An international treaty for seeking patent protection in multiple countries with a single application.
[7] Paris Convention for the Protection of Industrial Property (1883): An international treaty on the protection of industrial property rights.
[8] Berne Convention for the Protection of Literary and Artistic Works (1886): An international treaty for the protection of literary and artistic works.
[9] TRIPS Agreement (Agreement on Trade-Related Aspects of Intellectual Property Rights): Administered by the World Trade Organization (WTO), it sets minimum standards for intellectual property regulation.
[10] Mental Health Parity and Addiction Equity Act (MHPAEA), 2008: A U.S. federal law ensuring equal benefits for mental and substance use treatments as for medical/surgical care
[1R] Schneider T R, Lyons J B, Khazon S. Emotional intelligence and resilience. Pers Individ Dif. 2013 November; 55(8):909-14.
[2R] Maulding W S. Emotional Intelligence and Successful Leadership. In: Goleman D, editor. Kansas City: Information Analyses (070) Speeches/Meeting Papers (150); 2002. p. 1-15.
[3R] Chopra P K, Kanji G K. Emotional intelligence: A catalyst for inspirational leadership and management excellence. Total Quality Management & Business Excellence. 2010 October; 21(10):971-1004.
[4R] Grant L, Kinman G. Emotional Resilience in the Helping Professions and how it can be Enhanced. Health and Social Care Education. 2014 March; 3(1):23-34.
[5R] Bailen N H, Green L M, Thompson R J. Understanding Emotion in Adolescents: A Review of Emotional Frequency, Intensity, Instability, and Clarity. Emotion Review. 2019 Jan. 2; 11(1):63-73.
[6R] Mayer J D, Roberts R D, Barsade S G. Human Abilities: Emotional Intelligence. Annu Rev Psychol. 2008 Jan. 1; 59(1):507-36.
[7R] Butler A B. Triune Brain Concept: A Comparative Evolutionary Perspective. In: Encyclopedia of Neuroscience. Elsevier; 2009. p. 1185-93.
[8R] Kukreja P, Saxena K, Santra S. Edition 62-Mental Health Driving Change: Addressing America's Mental Health Crisis through the Power of Mobile Clinics in Underserved Communities. HPHR Journal. 2023; (62).
[9R] Das S. Depression in Working Professionals-A Systematic Review of the Literature. Scholars Journal of Applied Medical Sciences. 2023 Nov. 5; 11(11):1866-72.
[10R] Salleh M R. Life event, stress and illness. Malays J Med Sci. 2008 October; 15(4):9-18.
[11R] Peterson C T, Bauer S M, Chopra D, Mills P J, Maturi R K. Effects of Shambhavi Mahamudra Kriya, a Multicomponent Breath-Based Yogic Practice (Pranayama), on Perceived Stress and General Well-Being. J Evid Based Complementary Altern Med. 2017 Oct. 22; 22(4):788-97.
[12R] Davis M C. Building Emotional Resilience to Promote Health. Am J Lifestyle Med. 2009 Jul. 22; 3(1_suppl):60S-63S.
[13R] 2023 Work in America Survey [Internet]. 2023 April [cited 2023 Nov. 5]. Available from: https://www.apa.org/pubs/reports/work-in-America/2023-workplace-health-well-being
[14R] Glazer S, Liu C. Work, Stress, Coping, and Stress Management. In: Oxford Research Encyclopedia of Psychology. Oxford University Press; 2017.
[15R] Murden F, Bailey D, Mackenzie F, Oeppen R S, Brennan P A. The impact and effect of emotional resilience on performance: an overview for surgeons and other healthcare professionals. British Journal of Oral and Maxillofacial Surgery. 2018 November; 56(9):786-90.
[16R] Kahneman D. Thinking, Fast and Slow [Internet]. [cited 2023 Nov. 4]. Available from: https://www.academia.edu/9151361/Thinking_Fast_and_Slow
[17R] Egana-delSol P, Sun X, Sajda P. Neurophysiological markers of emotion regulation predict efficacy of entrepreneurship education. Sci Rep. 2023 May 3; 13(1):7206.
[18R] Fletcher D, Sarkar M. A grounded theory of psychological resilience in Olympic champions. Psychol Sport Exerc. 2012 September; 13(5):669-78.
[19R] Crum A J, Akinola M, Martin A, Fath S. The role of stress mindset in shaping cognitive, emotional, and physiological responses to challenging and threatening stress. Anxiety Stress Coping. 2017 Jul. 4; 30(4):379-95.
[20R] Beer M. High commitment high performance: How to build a resilient organization for sustained advantage. John Wiley\& Sons; 2009. 18-20 p.
[21R] González M F. Precarity for the global talent: The impact of visa policies on high-skilled immigrants' work in the United States. International Migration. 2022 Apr. 24; 60(2):193-207.
[22R] Mudholkar R. A New Matrix For Evaluating Original Contributions In Business [Internet]. 2023 [cited 2023 Nov. 15]. Available from: https://www.forbes.com/sites/forbesbusinesscouncil/2023/11/15/a-new-matrix-for-evaluating-original-contributions-in-business/amp/
[23R] Agarwal S. The Guiding Star: The Journey of an International Physician From Maternal Fetal Medicine to Fetal Neonatal Neurology. Child Neurol Open. 2021 Jan. 1; 8:2329048X2110307.
[24R] Seven Stars Unlock Their Extraordinary Ability to Earn a US Green Card in 6-Months of 2023. ACN Newswire. 2023 Sep. 25;
[25R] Goleman D. Leadership_The Power of Emotional Intelligence.

1. A method for facilitating assessing users, the method comprising:
transmitting, using a communication device, at least one prompt information of at least one prompt to at least one device, wherein the at least one device comprises at least one output device, wherein the at least one device is configured for presenting the at least one prompt to at least one user based on the at least one prompt information, wherein the at least one prompt information comprises at least one questionnaire, wherein the at least one questionnaire comprises at least one question and a plurality of answer options for each of the at least one question;
receiving, using the communication device, at least one response of the at least one user for the at least one prompt from the at least one device, wherein the at least one response comprises a selection of answer option from the plurality of answer options for at least one of the at least one question, wherein the at least one device further comprises a motion sensor, wherein the motion sensor is configured for detecting at least one of a gesture and a movement, wherein the motion sensor is configured for generating the at least one response based on the detecting;
detecting, using at least one sensor comprising at least one a pupilometer and a galvanic skin response (GSR) sensor, a physiological response of the at least one user for the at least one question;
generating, using a processing device, at least one sensor data for the at least one question based on the detecting of the physiological response;
analyzing, using the processing device, the at least one sensor data;
determining, using the processing device, a validity of each of the at least one response based on the analyzing of the at least one sensor data;
analyzing, using the processing device, the validity of each of the at least one response, wherein the validity indicates genuineness of the at least one response;
analyzing, using the processing device, the at least one response using at least one algorithm;
generating, using the processing device, at least one score for at least one metric based on the analyzing of the at least one response, and the analyzing of the validity of each of the at least one response, wherein the at least one metric comprises an Emotional Resilience and Motivation Quotient (ERMQ) metric, wherein the at least one score comprises an ERMQ score, wherein the at least one score comprises ERMQ score, wherein the ERMQ score ranges from 0 to 100, wherein the at least one score for the at least one metric quantifies a resilience capacity of the at least one user;
generating, using the processing device, at least one resilience profile for the at least one user based on the at least one score for the at least one metric;
transmitting, using the communication device, the at least one resilience profile to the at least one device; and
storing, using a storage device, at least one assessment data comprising the at least one response and the at least one score for the at least one metric, and the at least one resilience profile.

2. The method of claim 1 further comprising:
analyzing, using the processing device, the at least one response and the at least one resilience profile using at least one machine learning model, wherein the at least one machine learning model comprises at least one gradient-boosting decision tree model, wherein the at least one gradient-boosting decision tree model is trained on aggregated assessment data to generate personalize recommendation tailored to the at least one resilience profile of the at least one user;
generating, using the processing device, at least one recommendation for the at least one user based on the analyzing of the at least one response and the at least one resilience profile using the at least one machine learning model, wherein the at least one recommendation comprises a personalized guidance for the at least one user; and
transmitting, using the communication device, the at least one recommendation to the at least one device.

3. The method of claim 2, wherein the at least one machine learning model is an ensemble of at least 100 decision trees, wherein a maximum decision tree depth for the at least one machine learning model is at least 15, wherein the ensemble of at least 100 decision trees is trained using grid search hyperparameter optimization, wherein a training process associated with the at least one machine learning model evolves the at least one machine learning model to learn non-linear relationships and interactions between assessment attributes and optimal recommendations.

4. The method of claim 2 further comprising:
retrieving, using the storage device, at least one of a plurality of historical assessment data associated with a time duration after elapsing of the time duration; and
performing, using the processing device, an incremental training of the at least one machine learning model using at least one of the plurality of historical assessment data, wherein the analyzing of the at least one response and the at least one resilience profile using the at least one machine learning model is further based on the performing of the incremental training of the at least one machine learning model.

5. The method of claim 1 further comprising:
retrieving, using the storage device, a plurality of responses for a plurality of prompts associated with a plurality of users;
performing, using the processing device, a statistical modeling on the plurality of responses for determining a plurality of psychological skill attributes using a factor analysis;
performing, using the processing device, a regression modeling on the plurality of psychological skill attributes for determining a weight for each of the plurality of psychological skill attributes; and
generating, using the processing device, the at least one algorithm based on the performing of the statistical modeling and the performing of the regression modeling.

6. The method of claim 5, wherein the analyzing of the at least one response using the at least one algorithm comprises:
evaluating a competency of the at least one user against each of the plurality of psychological skill attributes based on the at least one response;
scoring each of the plurality of psychological skill attributes based on the evaluating; and
computing a weighted average score for the plurality of psychological skill attributes based on the weight of each of the plurality of psychological skill attributes and the scoring, wherein the generating of the at least one score for the at least one metric is further based on the computing.

7. The method of claim 6 further comprising:
obtaining, using the processing device, at least one data associated with the at least one user;
analyzing, using the processing device, the at least one data;
determining, using the processing device, a context associated with the assessing of the at least one user;
modifying, using the processing device, the weight associated with at least one of the plurality of psychological skill attributes based on the context; and
generating, using the processing device, a modified weight for at least one of the plurality of psychological skill attributes based on the modifying, wherein the computing of the weighted average score for the plurality of psychological skill attributes is further based on the modified weight of at least one of the plurality of psychological skill attributes.

8. The method of claim 1 further comprising:
obtaining, using the processing device, at least one data associated with the at least one user;
analyzing, using the processing device, the at least one data;
determining, using the processing device, a context associated with the assessing of the at least one user; and
generating, using the processing device, the at least one prompt information for the at least one prompt based on the determining of the context.

9. (canceled)

10. The method of claim 1, wherein the analyzing of the at least one response further comprises analyzing the at least one response using at least one behavioral model and at least one natural language processing (NLP) model, wherein the at least one behavioral model and the at least one NLP model are separately trained on a plurality of training responses, wherein the analyzing of the at least one response using the at least one behavioral model and the at least one NLP model comprises:
obtaining at least one first output from the at least one behavioral model by inputting the at least one response to the at least one behavioral model;
obtaining at least one second output from the at least one NLP model by inputting the at least one response to the at least one NLP model; and
combining the at least one first output and the at least one second output, wherein the generating of the at least one score for the at least one metric is further based on the combining.

11. A system for facilitating assessing users, the system comprising:
a communication device configured for:
transmitting at least one prompt information of at least one prompt to at least one device, wherein the at least one device comprises at least one output device, wherein the at least one device is configured for presenting the at least one prompt to at least one user based on the at least one prompt information, wherein the at least one prompt information comprises at least one questionnaire, wherein the at least one questionnaire comprises at least one question and a plurality of answer options for each of the at least one question;
receiving at least one response of the at least one user for the at least one prompt from the at least one device, wherein the at least one response comprises a selection of answer option from the plurality of answer options for at least one of the at least one question, wherein the at least one device further comprises a motion sensor, wherein the motion sensor is configured for detecting at least one of a gesture and a movement, wherein the motion sensor is configured for generating the at least one response based on the detecting; and
transmitting at least one profile to the at least one device;
at least one sensor comprising at least one a pupilometer and a galvanic skin response (GSR) sensor is configured for detecting a physiological response of the at least one user for the at least one question;
a processing device communicatively coupled with the communication device, wherein the processing device is configured for:
generating at least one sensor data for the at least one question based on the detecting of the physiological response;
analyzing the at least one sensor data;
determining a validity of each of the at least one response based on the analyzing of the at least one sensor data;
analyzing the validity of each of the at least one response, wherein the validity indicates genuineness of the at least one response;
analyzing the at least one response using at least one algorithm;
generating at least one score for at least one metric based on the analyzing of the at least one response, and the analyzing of the validity of each of the at least one response, wherein the at least one metric comprises an Emotional Resilience and Motivation Quotient (ERMQ) metric, wherein the at least one score comprises an ERMQ score, wherein the at least one score comprises ERMQ score, wherein the ERMQ score ranges from 0 to 100, wherein the at least one score for the at least one metric quantifies a resilience capacity of the at least one user; and
generating the at least one resilience profile for the at least one user based on the at least one score for the at least one metric; and
a storage device communicatively coupled with the processing device, wherein the storage device is configured for storing at least one assessment data comprising the at least one response and the at least one score for the at least one metric, and the at least one resilience profile.

12. The system of claim 11, wherein the processing device is further configured for:
analyzing the at least one response and the at least one resilience profile using at least one machine learning model, wherein the at least one machine learning model comprises at least one gradient-boosting decision tree model, wherein the at least one gradient-boosting decision tree model is trained on aggregated assessment data to generate personalize recommendation tailored to the at least one resilience profile of the at least one user; and
generating at least one recommendation for the at least one user based on the analyzing of the at least one response and the at least one resilience profile using the at least one machine learning model, wherein the at least one recommendation comprises a personalized guidance for the at least one user, wherein the storage device is further configured for transmitting the at least one recommendation to the at least one device.

13. The system of claim 12, wherein the at least one machine learning model is an ensemble of at least 100 decision trees, wherein a maximum decision tree depth for the at least one machine learning model is at least 15, wherein the ensemble of at least 100 decision trees is trained using grid search hyperparameter optimization, wherein a training process associated with the at least one machine learning model evolves the at least one machine learning model to learn non-linear relationships and interactions between assessment attributes and optimal recommendations.

14. The system of claim 12, wherein the storage device is further configured for retrieving at least one of a plurality of historical assessment data associated with a time duration after elapsing of the time duration, wherein the processing device is further configured for performing an incremental training of the at least one machine learning model using at least one of the plurality of historical assessment data, wherein the analyzing of the at least one response and the at least one resilience profile using the at least one machine learning model is further based on the performing of the incremental training of the at least one machine learning model.

15. The system of claim 11, wherein the storage device is further configured for retrieving a plurality of responses for a plurality of prompts associated with a plurality of users, wherein the processing device is further configured for:
performing a statistical modeling on the plurality of responses for determining a plurality of psychological skill attributes using a factor analysis;
performing a regression modeling on the plurality of psychological skill attributes for determining a weight for each of the plurality of psychological skill attributes; and
generating the at least one algorithm based on the performing of the statistical modeling and the performing of the regression modeling.

16. The system of claim 15, wherein the analyzing of the at least one response using the at least one algorithm comprises:
evaluating a competency of the at least one user against each of the plurality of psychological skill attributes based on the at least one response;
scoring each of the plurality of psychological skill attributes based on the evaluating; and
computing a weighted average score for the plurality of psychological skill attributes based on the weight of each of the plurality of psychological skill attributes and the scoring, wherein the generating of the at least one score for the at least one metric is further based on the computing.

17. The system of claim 16, wherein the processing device is further configured for:
obtaining at least one data associated with the at least one user;
analyzing the at least one data;
determining a context associated with the assessing of the at least one user; and
modifying the weight associated with at least one of the plurality of psychological skill attributes based on the context; and
generating a modified weight for at least one of the plurality of psychological skill attributes based on the modifying, wherein the computing of the weighted average score for the plurality of psychological skill attributes is further based on the modified weight of at least one of the plurality of psychological skill attributes.

18. The system of claim 11, wherein the processing device is further configured for:
obtaining at least one data associated with the at least one user;
analyzing the at least one data;
determining a context associated with the assessing of the at least one user; and
generating the at least one prompt information for the at least one prompt based on the determining of the context.

19. (canceled)

20. The system of claim 11, wherein the analyzing of the at least one response further comprises analyzing the at least one response using at least one behavioral model and at least one natural language processing (NLP) model, wherein the at least one behavioral model and the at least one NLP model are separately trained on a plurality of training responses, wherein the analyzing of the at least one response using the at least one behavioral model and the at least one NLP model comprises:
obtaining at least one first output from the at least one behavioral model by inputting the at least one response to the at least one behavioral model;
obtaining at least one second output from the at least one NLP model by inputting the at least one response to the at least one NLP model; and
combining the at least one first output and the at least one second output, wherein the generating of the at least one score for the at least one metric is further based on the combining.
